(19) United States
(12) Patent Application Publication
(10) Pub. No.: US 2026/0041633 A1
(43) Pub. Date: Feb. 12, 2026

(54) ANTI-TIGIT ANTIBODY FORMULATION

(71) Applicant: Compugen Ltd., Holon (IL)

(72) Inventors: Adeboye Henry Adewoye, MD, San Francsico, CA (US); Rafi Mohammad, Berkeley, CA (US); Wei Wang, Berkeley, CA (US)

(21) Appl. No.: 18/868,661

(22) PCT Filed: May 24, 2023
(86) PCT No.: PCT/US2023/067432

§ 371 Date: Nov. 22, 2024

Related U.S. Application Data
(60) Provisional application No. 63/346,167, filed on May 26, 2022.

(51) Int. Cl. A61K 9/08; A61K 9/00; A61K 9/19; A61K 39/00; A61K 47/18; A61K 47/20; A61K 47/22; A61K 47/26; C07K 16/28

(52) U.S. Cl. CPC A61K 9/08; A61K 9/0019; A61K 9/19; A61K 47/183; A61K 47/20; A61K 47/22; A61K 47/26; C07K 16/2803; A61K 2039/545

(57) ABSTRACT

The present invention provides formulations comprising anti-TIGIT antibodies with CDRs identical to those shown in FIG. 3 (i.e., CDRs identical to those for CPA.9.086.H4(S241P), as described herein.

### CROSS-REFERENCE TO RELATED APPLICATIONS

This application claims priority to U.S. Provisional Patent Application No. 63/346,167, filed on May 26, 2022, which is hereby incorporated by reference in its entirety.

### SEQUENCE LISTING

The instant application contains a Sequence Listing which has been submitted electronically in XML format and is hereby incorporated by reference in its entirety. Said XML copy, created on Sep. 16, 2025, is named 114386-5021-US_SL.xml and is 14,000 bytes in size.

### BACKGROUND OF THE INVENTION

TIGIT is a coinhibitory receptor that is highly expressed on effector & regulatory (Treg) CD4+ T cells, effector CD8+ T cells, and NK cells. TIGIT has been shown to attenuate immune response by (1) direct signaling, (2) inducing ligand signaling, and (3) competition with and disruption of signaling by the costimulatory receptor CD226 (also known as DNAM-1). TIGIT signaling has been the most well-studied in NK cells, where it has been demonstrated that engagement with its cognate ligand, poliovirus receptor (PVR, also known as CD155) directly suppresses NK cell cytotoxicity through its cytoplasmic ITIM domain. Knockout of the TIGIT gene or antibody blockade of the TIGIT/PVR interaction has shown to enhance NK cell killing in vitro, as well as to exacerbate autoimmune diseases in vivo. In addition to its direct effects on T- and NK cells, TIGIT can induce PVR-mediated signaling in dendritic or tumor cells, leading to the increase in production of anti-inflammatory cytokines such as IL10. In T-cells TIGIT can also inhibit lymphocyte responses by disrupting homodimerization of the costimulatory receptor CD226, and by competing with it for binding to PVR.

TIGIT is highly expressed on lymphocytes, including Tumor Infiltrating Lymphocytes (TILs) and Tregs, that infiltrate different types of tumors. PVR is also broadly expressed in tumors, suggesting that the TIGIT-PVR signaling axis may be a dominant immune escape mechanism for cancer. Notably, TIGIT expression is tightly correlated with the expression of another important coinhibitory receptor, PD1. TIGIT and PD1 are co-expressed on the TILs of numerous human and murine tumors. Unlike TIGIT and CTLA4, PD1 inhibition of T cell responses does not involve competition for ligand binding with a costimulatory receptor.

It is an object of the invention to provide formulations comprising anti-TIGIT antibodies with CDRs identical to those shown in FIG. 3 (i.e., CDRs identical to those for CPA.9.086.H4(S241P).

### BRIEF SUMMARY OF THE INVENTION

In some aspects, invention provides a stable liquid pharmaceutical formulation of an anti-TIGIT antibody comprising:
(a) an anti-TIGIT antibody, the anti-TIGIT antibody comprising:
   i) a heavy chain variable domain comprising the vhCDR1, vhCDR2, and vhCDR3 from the heavy chain of CPA.9.086.H4(S241P) (SEQ ID NO:5); and
   ii) a light chain variable domain comprising the vlCDR1, vlCDR2, and vlCDR3 from the light chain of CPA.9.086.H4(S241P) (SEQ ID NO:10);
(b) from 8 mM to 12 mM histidine;
(c) from 25 mM to 35 mM L-Arginine;
(d) from 8 mM to 12 mM L-methionine;
(e) from 7% (w/v) to 9% (w/v) sucrose; and
(f) from 70 ppm to 80 ppm polysorbate 80,
wherein the pharmaceutical formulation has a pH of 5.4+/−0.2.

In some embodiments of paragraph [0004], said anti-TIGIT antibody comprises a CH1-hinge-CH2-CH3 sequence of IgG4, wherein said hinge region optionally comprises mutations.

In some embodiments of paragraphs [0004]-[0005], said anti-TIGIT antibody comprises a CH1-hinge-CH2-CH3 sequence of IgG4, wherein said hinge region optionally comprises mutations.

In some embodiments of paragraphs [0004]-[0006], said heavy chain variable domain is from the heavy chain of CPA.9.086.H4(S241P) (SEQ ID NO:1) and the light chain variable domain is from the light chain of CPA.9.086.H4(S241P) (SEQ ID NO:6).

In some embodiments of paragraphs [0004]-[0007], said anti-TIGIT antibody comprises a CL region of human kappa 2 light chain.

In some embodiments of paragraphs [0004]-[0008], said pharmaceutical formulation comprises from about 15 mg/mL to about 25 mg/mL anti-TIGIT antibody.

In some embodiments of paragraphs [0004]-[0009], said pharmaceutical formulation comprises about 20 mg/mL anti-TIGIT antibody.

In some embodiments of paragraphs [0004]-[0010], said pharmaceutical formulation comprises about 10 mM histidine.

In some embodiments of paragraphs [0004]-[0011], said pharmaceutical formulation comprises about 30 mM L-arginine.

In some embodiments of paragraphs [0004]-[0012], said pharmaceutical formulation comprises about 10 mM L-methionine.

In some embodiments of paragraphs [0004]-[0013], said pharmaceutical formulation comprises about 8% sucrose.

In some embodiments of paragraphs [0004]-[0014], said pharmaceutical formulation comprises about 75 ppm polysorbate 80.

In some aspects, invention provides a stable liquid pharmaceutical formulation of an anti-TIGIT antibody comprising:
(a) about 20 mg/mL of an anti-TIGIT antibody, the anti-TIGIT antibody comprising:
   i) a heavy chain variable domain comprising the vhCDR1, vhCDR2, and vhCDR3 from the heavy chain of CPA.9.086.H4(S241P) (SEQ ID NO:1); and
   ii) a light chain variable domain comprising the vlCDR1, vlCDR2, and vlCDR3 from the light chain of CPA.9.086.H4(S241P) (SEQ ID NO:6);
(b) about 10 mM histidine;
(c) about 30 mM L-Arginine;
(d) about 10 mM L-methionine;
(e) about 8% (w/v) sucrose; and
(f) about 75 ppm polysorbate 80,
wherein the pharmaceutical formulation has a pH of 5.4+/−0.2.

In some aspects, invention provides a stable liquid pharmaceutical formulation of an anti-TIGIT antibody comprising:
(a) about 20 mg/mL of an anti-TIGIT antibody, the anti-TIGIT antibody comprising:
   i) a heavy chain variable domain from the heavy chain of CPA.9.086.H4(S241P) (SEQ ID NO:1); and
   ii) a light chain variable domain from the light chain of CPA.9.086.H4(S241P) (SEQ ID NO:6);
(b) about 10 mM histidine;
(c) about 30 mM L-Arginine;
(d) about 10 mM L-methionine;
(e) about 8% (w/v) sucrose; and
(f) about 75 ppm polysorbate 80, wherein the pharmaceutical formulation has a pH of 5.4+/−0.2.

In some aspects, invention provides a stable liquid pharmaceutical formulation of an anti-TIGIT antibody comprising:
(a) about 20 mg/mL of an anti-TIGIT antibody, the anti-TIGIT antibody comprising:
   i) a heavy chain from CPA.9.086.H4(S241P) (SEQ ID NO:5); and
   ii) a light chain from the light chain of CPA.9.086.H4(S241P) (SEQ ID NO: 10);
(b) about 10 mM histidine;
(c) about 30 mM L-Arginine;
(d) about 10 mM L-methionine;
(e) about 8% (w/v) sucrose; and
(f) about 75 ppm polysorbate 80,
wherein the pharmaceutical formulation has a pH of 5.4+/−0.2.

In some embodiments of paragraphs [0017]-[0018], said pharmaceutical formulation is stable at 5° C. to 25° C. for at least 1 week, 2 weeks, 3 weeks, 4 weeks, 2 months, 3 months, 4 months, 5 months, 6 months, 7 months, or 8 months.

In some embodiments of paragraphs [0017]-[0018], said pharmaceutical formulation is stable at 5° C. to 25° C. for at least 6 months, at least 12 months, or at least 18 months.

In some embodiments of paragraphs [0004]-[0020], said pharmaceutical formulation is administered at a dosage of from about 0.01 mg/kg to about 100 mg/kg, from about 0.05 mg/kg to about 50 mg/kg, from about 0.1 mg/kg to about 20 mg/kg, or from about 1 mg/kg to about 10 mg/kg.

In some embodiments of paragraphs [0004]-[0021], said pharmaceutical formulation is administered at a dosage of about 0.01 mg/kg, about 0.01 mg/kg, about 0.03 mg/kg, about 0.1 mg/kg, about 0.3 mg/kg, about 1 mg/kg, about 3 mg/kg, about 10 mg/kg, about 15 mg/kg, or about 20 mg/kg, or about 30 mg/kg.

In some embodiments of paragraphs [0004]-[0022], said anti-TIGIT antibody is administered once every week, once every two weeks, once every three weeks, or once every four weeks, once every five weeks, or once every six weeks.

In some embodiments of paragraphs [0004]-[0023], said anti-TIGIT antibody is administered as a single dose.

In some embodiments of paragraphs [0004]-[0024], said pharmaceutical formulation is formulated for intravenous (IV) infusion.

In some embodiments of paragraphs [0004]-[0025], said pharmaceutical formulation is administered for the treatment of cancer.

In some embodiments of paragraphs [0004]-[0026], said pharmaceutical formulation is for use in a method of treating cancer.

In some embodiments of paragraphs [0026]-[0027], said cancer is selected from the group consisting of prostate cancer, liver cancer (HCC), colorectal cancer (CRC), colorectal cancer MSS (MSS-CRC; including refractory MSS colorectal), CRC (MSS unknown), ovarian cancer (including ovarian carcinoma), endometrial cancer (including endometrial carcinoma), breast cancer, pancreatic cancer, stomach cancer, cervical cancer, head and neck cancer, thyroid cancer, testis cancer, urothelial cancer, lung cancer, melanoma, non-melanoma skin cancer (squamous and basal cell carcinoma), glioma, renal cell cancer (RCC), renal cell carcinoma (RCC), lymphoma (non-Hodgkins' lymphoma (NHL) and Hodgkin's lymphoma (HD)), Acute myeloid leukemia (AML), T cell Acute Lymphoblastic Leukemia (T-ALL), Diffuse Large B cell lymphoma, testicular germ cell tumors, mesothelioma, esophageal cancer, triple negative breast cancer, Merkel Cells cancer, MSI-high cancer, KRAS mutant tumors, adult T-cell leukemia/lymphoma, pleural mesothelioma, anal SCC, small cell lung cancer, adenocarcinoma of the lung, neuroendocrine lung cancer (including neuroendocrine lung carcinoma), HNSCC, PD1 refractory or relapsing HNSCC, NSCLC, PD1 refractory or relapsing NSCLC, NSCLC large cell, NSCLC squamous cell, cervical SCC, malignant melanoma, pancreatic cancer, pancreatic adenocarcinoma, NSCLC, adenoid cystic cancer (including adenoid cystic carcinoma), primary peritoneal cancer, microsatellite stable primary peritoneal cancer, platinum resistant microsatellite stable primary peritoneal cancer, multiple myeloma, gastroesophageal junction cancer, and/or Myelodysplastic syndromes (MDS).

In some embodiments, said formulation is obtained by lyophilizing said stable liquid pharmaceutical formulation according to any one of paragraphs [0004]-[0028].

In some embodiments, said use of a stable lyophilized pharmaceutical formulation according to any one of paragraphs [0004]-[0029] for the treatment of cancer.

In some embodiments of paragraphs [0030], said cancer is selected from the group consisting of prostate cancer, liver cancer (HCC), colorectal cancer (CRC), colorectal cancer MSS (MSS-CRC; including refractory MSS colorectal), CRC (MSS unknown), ovarian cancer (including ovarian carcinoma), endometrial cancer (including endometrial carcinoma), breast cancer, pancreatic cancer, stomach cancer, cervical cancer, head and neck cancer, thyroid cancer, testis cancer, urothelial cancer, lung cancer, melanoma, non-melanoma skin cancer (squamous and basal cell carcinoma), glioma, renal cell cancer (RCC), renal cell carcinoma (RCC), lymphoma (non-Hodgkins' lymphoma (NHL) and Hodgkin's lymphoma (HD)), Acute myeloid leukemia (AML), T cell Acute Lymphoblastic Leukemia (T-ALL), Diffuse Large B cell lymphoma, testicular germ cell tumors, mesothelioma, esophageal cancer, triple negative breast cancer, Merkel Cells cancer, MSI-high cancer, KRAS mutant tumors, adult T-cell leukemia/lymphoma, pleural mesothelioma, anal SCC, small cell lung cancer, adenocarcinoma of the lung, neuroendocrine lung cancer (including neuroendocrine lung carcinoma), HNSCC, PD1 refractory or relapsing HNSCC, NSCLC, PD1 refractory or relapsing NSCLC, NSCLC large cell, NSCLC squamous cell, cervical SCC, malignant melanoma, pancreatic cancer, pancreatic adenocarcinoma, NSCLC, adenoid cystic cancer (including adenoid cystic carcinoma), primary peritoneal cancer, microsatellite stable primary peritoneal cancer, platinum resistant microsatellite stable primary peritoneal cancer, multiple myeloma, gastroesophageal junction cancer, and/or Myelodysplastic syndromes (MDS).

### BRIEF DESCRIPTION OF THE DRAWINGS

FIG. 1 depicts the sequences of human and cynomolgus macaque (referred to as cyno) TIGIT ECD.

FIG. 2 depicts the sequences of human IgG4 and an alternate version of IgG4.

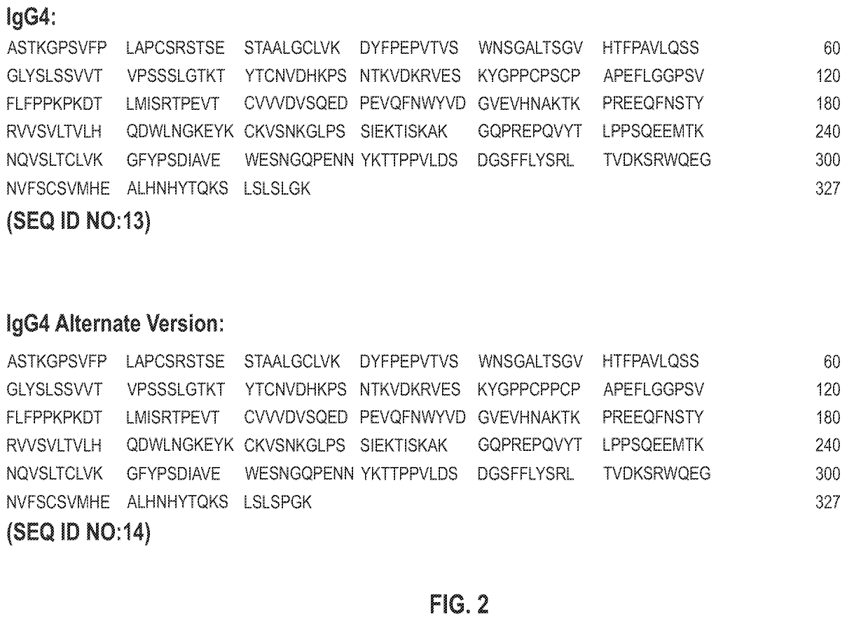

FIG. 3 depicts the sequences of anti-TIGIT antibody CPA.9.086 (S241P). Unless otherwise noted, the CDRs utilize the IMGT numbering (including the antibodies of the sequence listing.

FIG. 4 depicts SEC-HPLC separation of CPA.9.086.H4(S241P) monomer (peak 1) and its aggregates (peak 2) for the liquid stability sample.

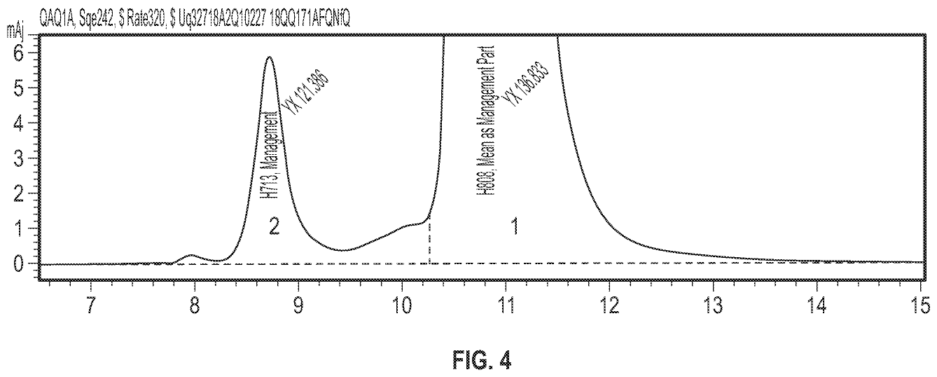

FIG. 5 depicts a representative electrophoretogram of the liquid stability sample, consisting of three regions-acidic (left), main (middle), and basic (right) regions.

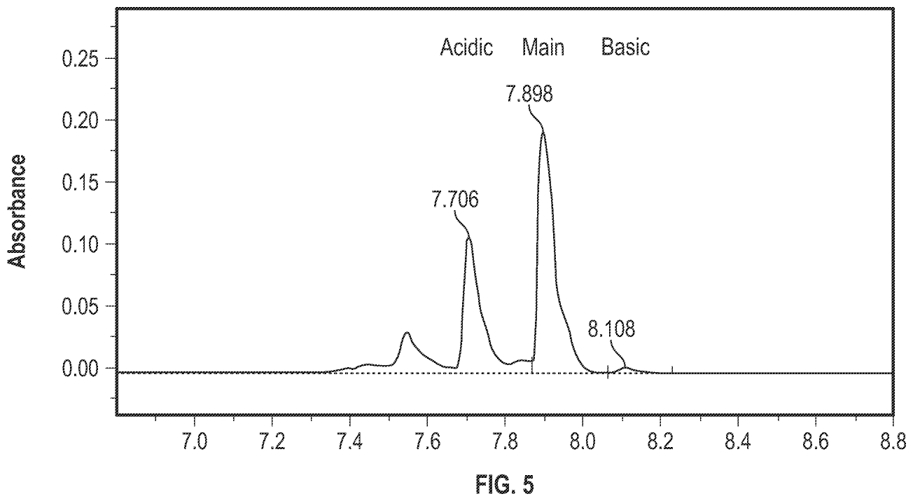

FIG. 6 provides photographs of formulation buffer (1) and CPA.9.086.H4(S241P) (2) in a 100 ml bottle (A) and a 10-mL glass vial (B).

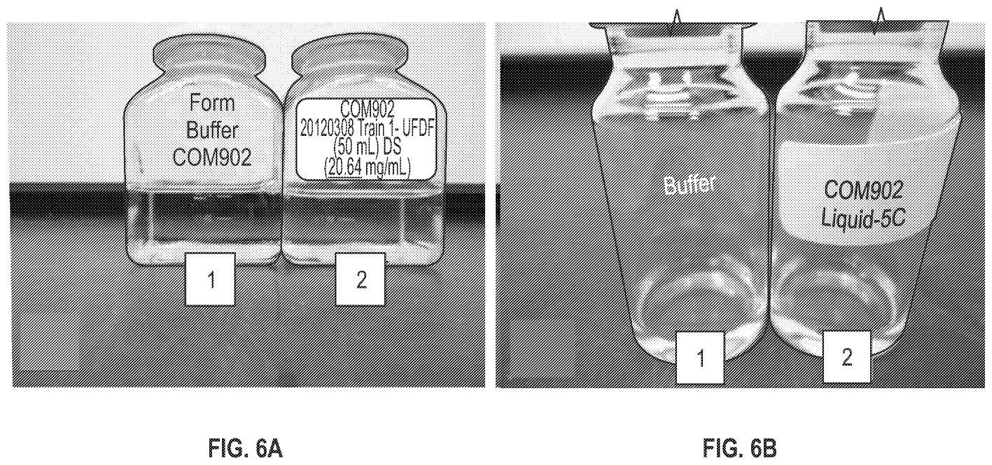

FIG. 7 provides photograph of lyophilized CPA.9.086.H4(S241P) in vials (A1 and A2) and reconstituted product vial (B2) in comparison with a buffer solution (B1).

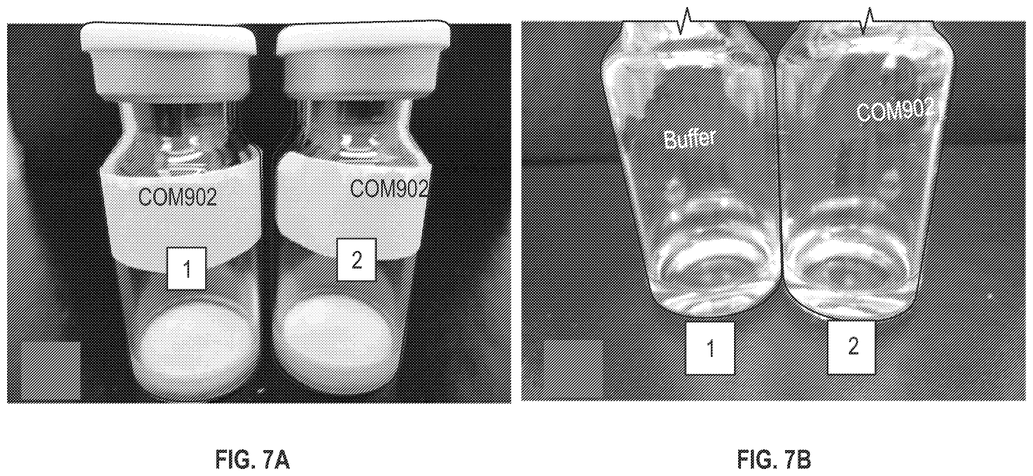

FIG. 8 provides photographs of 6-month liquid stability samples (A) and reconstituted lyophilized stability samples (B) at 5° C. (2) and 25° C. (3) in comparison with buffer (1).

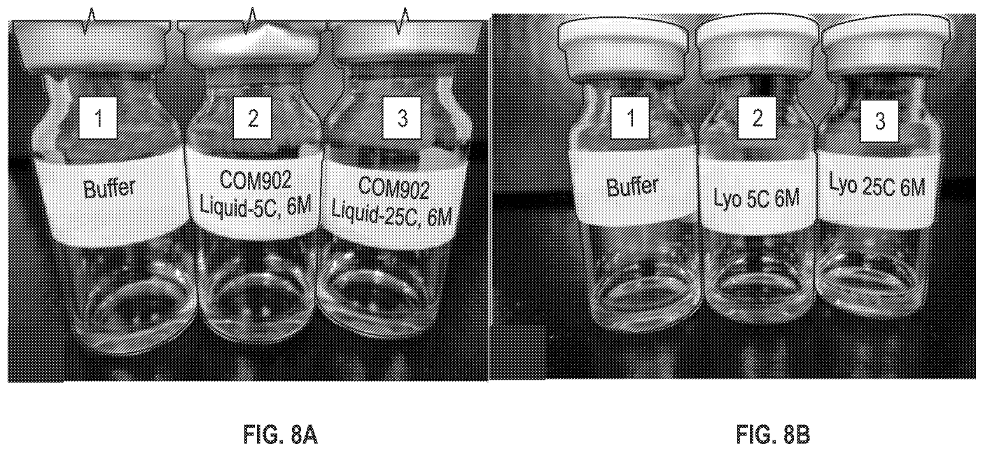

FIG. 9 provides a comparison of formulation buffer (left) with freeze-thawed samples at −20° C. and −70° C. Samples were freeze-thawed once (1×), 3 times (3×) and five times (5×).

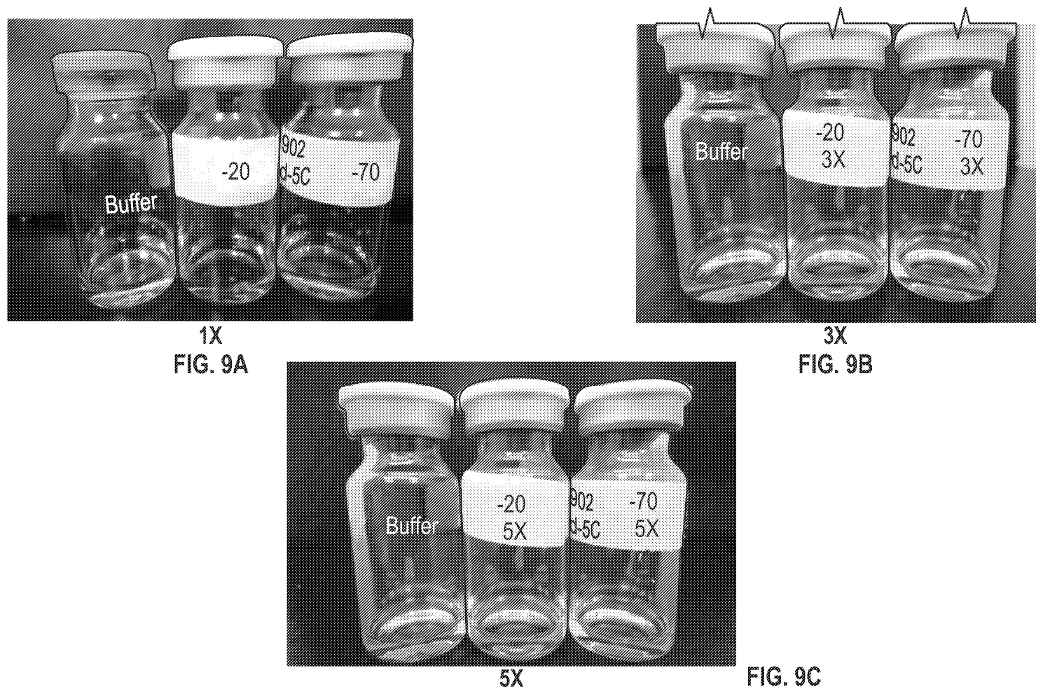

FIG. 10 provides a comparison of formulation buffer (left) with shaken samples (right) for 20 minutes (A), 6 hours (B) and 24 hours (C) at room temperature.

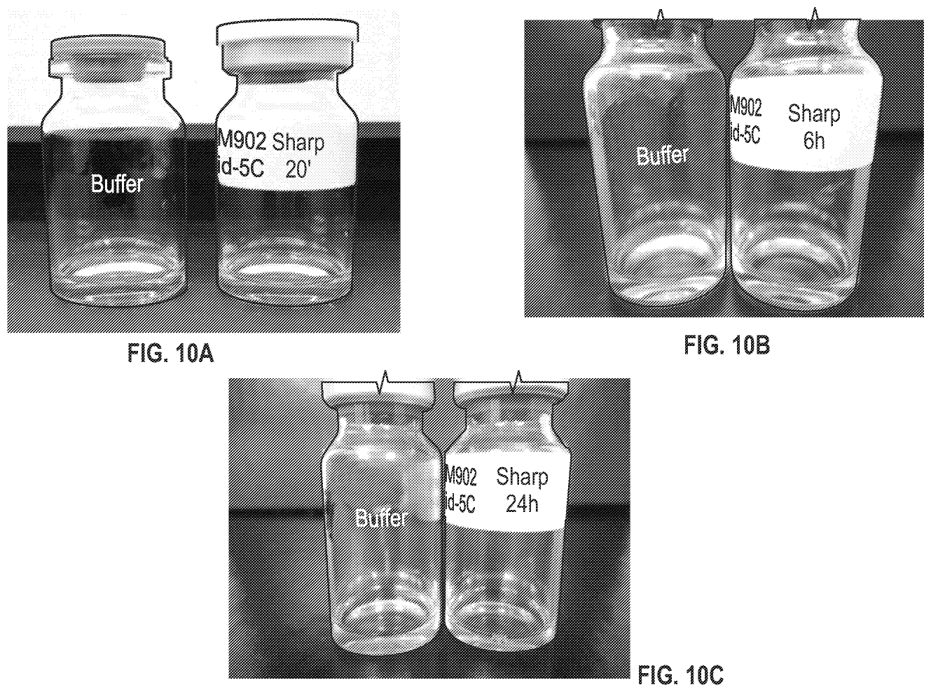

### DETAILED DESCRIPTION OF THE INVENTION

### I. Introduction

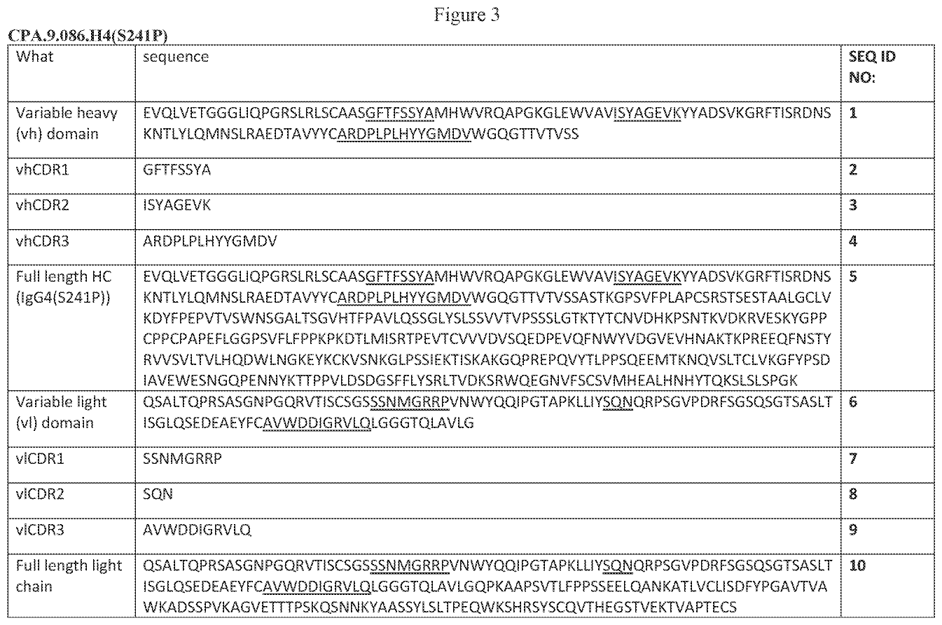

The present invention is directed to stable formulations anti-TIGIT antibody CPA.9.086 (S241P) (e.g., anti-TIGIT antibody with CDRs identical to those shown in FIG. 3) and provides these formulations. In particular, these formulations comprise antibodies comprising heavy and light chains as well as the vhCDR1, vhCDR2, vhCDR3, vlCDR1, vlCDR2 and vlCDR3 sequences from anti-TIGIT antibody CPA.9.086 (S241P). In some embodiments, the anti-TIGIT antibodies include those with CDRs identical to those shown in FIG. 3, as well as anti-TIGIT antibodies comprising the heavy and light chains as provided in FIG. 3.

### II. Tigit Proteins

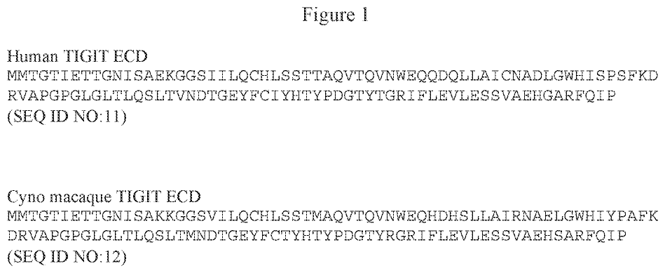

The present invention provides antibodies that specifically bind to TIGIT proteins and prevent activation by its ligand protein, PVR, poliovirus receptor (aka, CD155) a human plasma membrane glycoprotein, wherein the anti-TIGIT antibody is CPA.9.086 (S241P) or an anti-TIGIT antibody with CDRs identical to those shown in FIG. 3 (i.e., CDRs identical to those for CPA.9.086 (S241P). TIGIT, or T cell immunoreceptor with Ig and ITIM domains, is a co-inhibitory receptor protein also known as WUCAM, Vstm3 or Vsig9. TIGIT has an immunoglobulin variable domain, a transmembrane domain, and an immunoreceptor tyrosine-based inhibitory motif (ITIM) and contains signature sequence elements of the PVR protein family. The extracellular domain (ECD) sequences of TIGIT and of PVR are shown in FIG. 1. The antibodies formulated according to the invention include those that are specific for the TIGIT ECD such that the binding of TIGIT and PVR is blocked, for example an anti-TIGIT antibodies with CDRs identical to those shown in FIG. 3 (i.e., CDRs identical to those for CPA.9.086.H4(S241P).

Accordingly, as used herein, the term “TIGIT” or “TIGIT protein” or “TIGIT polypeptide” may optionally include any such protein, or variants, conjugates, or fragments thereof, including but not limited to known or wild type TIGIT, as described herein, as well as any naturally occurring splice variants, amino acid variants or isoforms, and in particular the ECD fragment of TIGIT.

### III. Anti-Tigit Antibodies

Accordingly, the invention provides anti-TIGIT antibodies with CDRs identical to those shown in FIG. 3 (i.e., CDRs identical to those for CPA.9.086.H4(S241P) that can be formulated according to the formulations described herein.

As is discussed below, the term “antibody” is used generally. Antibodies that find use in the present invention can take on a number of formats as described herein, including traditional antibodies as well as antibody derivatives, fragments and mimetics, described below. In general, the term “antibody” includes any polypeptide that includes at least one antigen binding domain, as more fully described below. Antibodies may be polyclonal, monoclonal, xenogeneic, allogeneic, syngeneic, or modified forms thereof, as described herein, with monoclonal antibodies finding particular use in many embodiments. In some embodiments, antibodies of the invention bind specifically or substantially specifically to TIGIT molecules. The terms “monoclonal antibodies” and “monoclonal antibody composition”, as used herein, refer to a population of antibody molecules that contain only one species of an antigen-binding site capable of immunoreacting with a particular epitope of an antigen, whereas the term “polyclonal antibodies” and “polyclonal antibody composition” refer to a population of antibody molecules that contain multiple species of antigen-binding sites capable of interacting with a particular antigen. A monoclonal antibody composition, typically displays a single binding affinity for a particular antigen with which it immunoreacts.

The amino-terminal portion of each chain includes a variable region of about 100 to 110 or more amino acids primarily responsible for antigen recognition, generally referred to in the art and herein as the “Fv domain” or “Fv region”. In the variable region, three loops are gathered for each of the V domains of the heavy chain and light chain to form an antigen-binding site. Each of the loops is referred to as a complementarity-determining region (hereinafter referred to as a “CDR”), in which the variation in the amino acid sequence is most significant. “Variable” refers to the fact that certain segments of the variable region differ extensively in sequence among antibodies. Variability within the variable region is not evenly distributed. Instead, the V regions consist of relatively invariant stretches called framework regions (FRs) of 15-30 amino acids separated by shorter regions of extreme variability called “hypervariable regions”.

Each VH and VL is composed of three hypervariable regions (“complementary determining regions,” “CDRs”) and four FRs, arranged from amino-terminus to carboxy-terminus in the following order: FR1-CDR1-FR2-CDR2-FR3-CDR3-FR4.

The hypervariable region generally encompasses amino acid residues from about amino acid residues 24-34 (LCDR1; “L” denotes light chain), 50-56 (LCDR2) and 89-97 (LCDR3) in the light chain variable region and around about 31-35B (HCDR1; “H” denotes heavy chain), 50-65 (HCDR2), and 95-102 (HCDR3) in the heavy chain variable region, although sometimes the numbering is shifted slightly as will be appreciated by those in the art; Kabat et al., SEQUENCES OF PROTEINS OF IMMUNOLOGICAL INTEREST, 5 th Ed. Public Health Service, National Institutes of Health, Bethesda, Md. (1991) and/or those residues forming a hypervariable loop (e.g., residues 26-32 (LCDR1), 50-52 (LCDR2) and 91-96 (LCDR3) in the light chain variable region and 26-32 (HCDR1), 53-55 (HCDR2) and 96-101 (HCDR3) in the heavy chain variable region; Chothia and Lesk (1987) J. Mol. Biol. 196:901-917. Specific CDRs of the invention are described below and shown in FIG. 3.

The carboxy-terminal portion of each chain defines a constant region primarily responsible for effector function. Kabat et al. collected numerous primary sequences of the variable regions of heavy chains and light chains. Based on the degree of conservation of the sequences, they classified individual primary sequences into the CDR and the framework and made a list thereof (see SEQUENCES OF IMMUNOLOGICAL INTEREST, 5th edition, NIH publication, No. 91-3242, E. A. Kabat et al., entirely incorporated by reference).

In the IgG subclass of immunoglobulins, there are several immunoglobulin domains in the heavy chain. By “immunoglobulin (Ig) domain” herein is meant a region of an immunoglobulin having a distinct tertiary structure. Of interest in the present invention are the heavy chain domains, including, the constant heavy (CH) domains and the hinge domains. In the context of IgG antibodies, the IgG isotypes each have three CH regions. Accordingly, “CH” domains in the context of IgG are as follows: “CH1” refers to positions 118-220 according to the EU index as in Kabat. “CH2” refers to positions 237-340 according to the EU index as in Kabat, and “CH3” refers to positions 341-447 according to the EU index as in Kabat.

Accordingly, the invention provides variable heavy domains, variable light domains, heavy constant domains, light constant domains and Fc domains to be used as outlined herein. By “variable region” as used herein is meant the region of an immunoglobulin that comprises one or more Ig domains substantially encoded by any of the Vκ or Vλ, and/or VH genes that make up the kappa, lambda, and heavy chain immunoglobulin genetic loci respectively. Accordingly, the variable heavy domain comprises vhFR1-vhCDR1-vhFR2-vhCDR2-vhFR3-vhCDR3-vhFR4, and the variable light domain comprises vlFR1-vlCDR1-vlFR2-vlCDR2-vlFR3-vlCDR3-vlFR4. By “heavy constant region” herein is meant the CH1-hinge-CH2-CH3 portion of an antibody. By “Fc” or “Fc region” or “Fc domain” as used herein is meant the polypeptide comprising the constant region of an antibody excluding the first constant region immunoglobulin domain and in some cases, part of the hinge. Thus, Fc refers to the last two constant region immunoglobulin domains of IgA, IgD, and IgG, the last three constant region immunoglobulin domains of IgE and IgM, and the flexible hinge N-terminal to these domains. For IgA and IgM, Fc may include the J chain. For IgG, the Fc domain comprises immunoglobulin domains Cγ2 and Cγ3 (Cγ2 and Cγ3) and the lower hinge region between Cγ1 (Cγ1) and Cγ2 (Cγ2). Although the boundaries of the Fc region may vary, the human IgG heavy chain Fc region is usually defined to include residues C226 or P230 to its carboxyl-terminus, wherein the numbering is according to the EU index as in Kabat. In some embodiments, as is more fully described below, amino acid modifications are made to the Fc region, for example to alter binding to one or more FcγR receptors or to the FcRn receptor.

Thus, “Fc variant” or “variant Fc” as used herein is meant a protein comprising an amino acid modification in an Fc domain. The Fc variants of the present invention are defined according to the amino acid modifications that compose them. Likewise, M428L/N434S defines an Fc variant with the substitutions M428L and N434S relative to the parent Fc polypeptide. The identity of the WT amino acid may be unspecified, in which case the aforementioned variant is referred to as 428L/434S. It is noted that the order in which substitutions are provided is arbitrary, that is to say that, for example, 428L/434S is the same Fc variant as M428L/N434S, and so on. For all positions discussed in the present invention that relate to antibodies, unless otherwise noted, amino acid position numbering is according to the EU index.

By “Fab” or “Fab region” as used herein is meant the polypeptide that comprises the VH, CH1, VL, and CL immunoglobulin domains. Fab may refer to this region in isolation, or this region in the context of a full-length antibody, antibody fragment or Fab fusion protein. By “Fv” or “Fv fragment” or “Fv region” as used herein is meant a polypeptide that comprises the VL and VH domains of a single antibody. As will be appreciated by those in the art, these generally are made up of two chains.

Throughout the present specification, either the IMTG numbering system or the Kabat numbering system is generally used when referring to a residue in the variable domain (approximately, residues 1-107 of the light chain variable region and residues 1-113 of the heavy chain variable region) (e.g., Kabat et al., supra (1991)). EU numbering as in Kabat is generally used for constant domains and/or the Fc domains.

Included within the definition of “antibody” is an “antigen-binding portion” of an antibody (also used interchangeably with “antigen-binding fragment”, “antibody fragment” and “antibody derivative”). That is, for the purposes of the invention, anti-TIGIT antibody with CDRs identical to those shown in FIG. 3 (i.e., CDRs identical to those for CPA.9.086.H4(S241P) of the invention has a minimum functional requirement that it bind to a TIGIT antigen. As will be appreciated by those in the art, there are a large number of antigen fragments and derivatives that retain the ability to bind an antigen and yet have alternative structures, including, but not limited to, (i) the Fab fragment consisting of VL, VH, CL and CH1 domains, (ii) the Fd fragment consisting of the VH and CH1 domains, (iii) F(ab′) 2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al., 1988, Science 242:423-426, Huston et al., 1988, Proc. Natl. Acad. Sci. U.S.A. 85:5879-5883, entirely incorporated by reference), (iv) “diabodies” or “triabodies”, multivalent or multispecific fragments constructed by gene fusion (Tomlinson et. al., 2000, Methods Enzymol. 326:461-479; WO94/13804; Holliger et al., 1993, Proc. Natl. Acad. Sci. U.S.A. 90:6444-6448, all entirely incorporated by reference), (v) “domain antibodies” or “dAb” (sometimes referred to as an “immunoglobulin single variable domain”, including single antibody variable domains from other species such as rodent (for example, as disclosed in WO 00/29004), nurse shark and Camelid V-HH dAbs, (vi) SMIPs (small molecule immunopharmaceuticals), camelbodies, nanobodies and IgNAR. The present invention is directed to anti-TIGIT antibodies with CDRs identical to those shown in FIG. 3 (i.e., CDRs identical to those for CPA.9.086.H4(S241P) that generally are based on the IgG class, which has several subclasses, including, but not limited to IgG4.

The amino-terminal portion of each chain includes a variable region of about 100 to 110 or more amino acids primarily responsible for antigen recognition, generally referred to in the art and herein as the “Fv domain” or “Fv region”. In the variable region, three loops are gathered for each of the V domains of the heavy chain and light chain to form an antigen-binding site. Each of the loops is referred to as a complementarity-determining region (hereinafter referred to as a “CDR”), in which the variation in the amino acid sequence is most significant. “Variable” refers to the fact that certain segments of the variable region differ extensively in sequence among antibodies. Variability within the variable region is not evenly distributed. Instead, the V regions consist of relatively invariant stretches called framework regions (FRs) of 15-30 amino acids separated by shorter regions of extreme variability called “hypervariable regions” that are each 9-15 amino acids long or longer.

Each VH and VL is composed of three hypervariable regions (“complementary determining regions,” “CDRs”) and four FRs, arranged from amino-terminus to carboxy-terminus in the following order: FR1-CDR1-FR2-CDR2-FR3-CDR3-FR4.

The hypervariable region generally encompasses amino acid residues from about amino acid residues 24-34 (LCDR1; “L” denotes light chain), 50-56 (LCDR2) and 89-97 (LCDR3) in the light chain variable region and around about 31-35B (HCDR1; “H” denotes heavy chain), 50-65 (HCDR2), and 95-102 (HCDR3) in the heavy chain variable region; Kabat et al., SEQUENCES OF PROTEINS OF IMMUNOLOGICAL INTEREST, 5th Ed. Public Health Service, National Institutes of Health, Bethesda, Md. (1991) and/or those residues forming a hypervariable loop (e.g. residues 26-32 (LCDR1), 50-52 (LCDR2) and 91-96 (LCDR3) in the light chain variable region and 26-32 (HCDR1), 53-55 (HCDR2) and 96-101 (HCDR3) in the heavy chain variable region; Chothia and Lesk (1987) J. Mol. Biol. 196:901-917. Specific CDRs of the invention are described below.

As will be appreciated by those in the art, the exact numbering and placement of the CDRs can be different among different numbering systems. However, it should be understood that the disclosure of a variable heavy and/or variable light sequence includes the disclosure of the associated (inherent) CDRs. Accordingly, the disclosure of each variable heavy region is a disclosure of the vhCDRs (e.g. vhCDR1, vhCDR2 and vhCDR3) and the disclosure of each variable light region is a disclosure of the vlCDRs (e.g. vlCDR1, vlCDR2 and vlCDR3). A useful comparison of CDR numbering is as below, see Lafranc et al., Dev. Comp. Immunol. 27 (1): 55-77 (2003):

| | Kabat + | | | | | |
|---|---|---|---|---|---|---|
| | Clothia | IMGT | Kabat | AbM | Chothia | Contact |
| vhCDR1 | 26-35 | 27-38 | 31-35 | 26-35 | 26-32 | 30-35 |
| vhCDR2 | 50-65 | 56-65 | 50-65 | 50-58 | 53-55 | 47-58 |
| vhCDR3 | 95-102 | 105-117 | 95-102 | 95-102 | 96-101 | 93-101 |
| vlCDR1 | 24-34 | 27-38 | 24-34 | 24-34 | 26-32 | 30-36 |
| vlCDR2 | 50-56 | 56-65 | 50-56 | 50-56 | 50-52 | 46-55 |
| vlCDR3 | 89-97 | 105-117 | 89-97 | 89-97 | 91-96 | 89-96 |

Throughout the present specification, the IMGT and/or Kabat numbering system is generally used when referring to a residue in the variable domain (approximately, residues 1-107 of the light chain variable region and residues 1-113 of the heavy chain variable region) and the hinge and the EU numbering system for Fc regions (e.g., Kabat et al., supra (1991)).

The present invention provides anti-TIGIT antibodies with CDRs identical to those shown in FIG. 3 (i.e., CDRs identical to those for CPA.9.086.H4(S241P). In this case, a “full CDR set” comprises the three variable light and three variable heavy CDRs, e.g., a vlCDR1, vlCDR2, vlCDR3, vhCDR1, vhCDR2 and vhCDR3 as provided in FIG. 3. These can be part of a larger variable light or variable heavy domain, respectfully. In addition, the variable heavy and variable light domains can be on separate polypeptide chains, when a heavy and light chain is used, or on a single polypeptide chain in the case of scFv sequences.

The CDRs contribute to the formation of the antigen-binding, or more specifically, epitope binding site of antibodies. “Epitope” refers to a determinant that interacts with a specific antigen binding site in the variable region of an antibody molecule known as a paratope. Epitopes are groupings of molecules such as amino acids or sugar side chains and usually have specific structural characteristics, as well as specific charge characteristics. A single antigen may have more than one epitope. The epitope may comprise amino acid residues directly involved in the binding (also called immunodominant component of the epitope) and other amino acid residues, which are not directly involved in the binding, such as amino acid residues which are effectively blocked by the specifically antigen binding peptide; in other words, the amino acid residue is within the footprint of the specifically antigen binding peptide. Epitopes may be either conformational or linear. A conformational epitope is produced by spatially juxtaposed amino acids from different segments of the linear polypeptide chain. A linear epitope is one produced by adjacent amino acid residues in a polypeptide chain. Conformational and non-conformational epitopes may be distinguished in that the binding to the former but not the latter is lost in the presence of denaturing solvents. An epitope typically includes at least 3, and more usually, at least 5 or 8-10 amino acids in a unique spatial conformation. Antibodies that recognize the same epitope can be verified in a simple immunoassay showing the ability of one antibody to block the binding of another antibody to a target antigen, for example “binning.”

Another type of Ig domain of the heavy chain is the hinge region. By “hinge” or “hinge region” or “antibody hinge region” or “immunoglobulin hinge region” herein is meant the flexible polypeptide comprising the amino acids between the first and second constant domains of an antibody. Structurally, the IgG CH1 domain ends at EU position 220, and the IgG CH2 domain begins at residue EU position 237. Thus, for IgG the antibody hinge is herein defined to include positions 221 to 236, wherein the numbering is according to the EU index as in Kabat.

The light chain generally comprises two domains, the variable light domain (containing the light chain CDRs and together with the variable heavy domains forming the Fv region), and a constant light chain region (often referred to as CL or CK). In general, either the constant lambda or constant kappa domain can be used, with lambda generally finding use in the invention.

In general, the anti-TIGIT antibodies with CDRs identical to those shown in FIG. 3 (i.e., CDRs identical to those for CPA.9.086.H4(S241P) of the invention are recombinant. “Recombinant” as used herein, refers broadly with reference to a product, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “recombinant antibody”, as used herein, includes all antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis to the non-CDR regions (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

### A. Chimeric and Humanized Anti-TIGIT Antibodies

In some embodiments, the anti-TIGIT antibodies with CDRs identical to those shown in FIG. 3 (i.e., CDRs identical to those for CPA.9.086.H4(S241P) can be derived from a mixture from different species, e.g., a chimeric antibody and/or a humanized antibody. In general, both “chimeric antibodies” and “humanized antibodies” refer to antibodies that combine regions from more than one species. For example, “chimeric antibodies” traditionally comprise variable region(s) from a mouse (or rat, in some cases) and the constant region(s) from a human. “Humanized antibodies” generally refer to non-human antibodies that have had the variable-domain framework regions swapped for sequences found in human antibodies. Generally, in a humanized antibody, the entire antibody, except the CDRs, is encoded by a polynucleotide of human origin or is identical to such an antibody except within its CDRs. The CDRs, some or all of which are encoded by nucleic acids originating in a non-human organism, are grafted into the beta-sheet framework of a human antibody variable region to create an antibody, the specificity of which is determined by the engrafted CDRs. The creation of such antibodies is described in, e.g., WO 92/11018, Jones, 1986, Nature 321:522-525, Verhoeyen et al., 1988, Science 239:1534-1536, all entirely incorporated by reference. “Backmutation” of selected acceptor framework residues to the corresponding donor residues is often required to regain affinity that is lost in the initial grafted construct (U.S. Pat. Nos. 5,530,101; 5,585,089; 5,693,761; 5,693,762; 6,180,370; 5,859,205; 5,821,337; 6,054,297; 6,407,213, all entirely incorporated by reference). The humanized antibody optimally also will comprise at least a portion, and usually all, of an immunoglobulin constant region, typically that of a human immunoglobulin, and thus will typically comprise a human Fc region. Humanized antibodies can also be generated using mice with a genetically engineered immune system. Roque et al., 2004, Biotechnol. Prog. 20:639-654, entirely incorporated by reference. A variety of techniques and methods for humanizing and reshaping non-human antibodies are well known in the art (See Tsurushita & Vasquez, 2004, Humanization of Monoclonal Antibodies, Molecular Biology of B Cells, 533-545, Elsevier Science (USA), and references cited therein, all entirely incorporated by reference). Humanization methods include but are not limited to methods described in Jones et al., 1986, Nature 321:522-525; Riechmann et al., 1988; Nature 332:323-329; Verhoeyen et al., 1988, Science, 239:1534-1536; Queen et al., 1989, Proc Natl Acad Sci, USA 86:10029-33; He et al., 1998, J. Immunol. 160:1029-1035; Carter et al., 1992, Proc Natl Acad Sci USA 89:4285-9, Presta et al., 1997, Cancer Res. 57 (20): 4593-9; Gorman et al., 1991, Proc. Natl. Acad. Sci. USA 88:4181-4185; O'Connor et al., 1998, Protein Eng 11:321-8, all entirely incorporated by reference. Humanization or other methods of reducing the immunogenicity of nonhuman antibody variable regions may include resurfacing methods, as described for example in Roguska et al., 1994, Proc. Natl. Acad. Sci. USA 91:969-973, entirely incorporated by reference.

Thus, the vhCDRs and vlCDRs from anti-TIGIT antibodies with CDRs identical to those shown in FIG. 3 (i.e., CDRs identical to those for CPA.9.086.H4(S241P) may be humanized (or “rehumanized”, for those that were already humanized).

In certain embodiments, the anti-TIGIT antibodies with CDRs identical to those shown in FIG. 3 (i.e., CDRs identical to those for CPA.9.086.H4(S241P) comprise a heavy chain variable region from a particular germline heavy chain immunoglobulin gene and/or a light chain variable region from a particular germline light chain immunoglobulin gene. For example, such antibodies may comprise or consist of a human antibody comprising heavy or light chain variable regions that are “the product of” or “derived from” a particular germline sequence. A human antibody that is “the product of” or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody. A human antibody that is “the product of” or “derived from” a particular human germline immunoglobulin sequence may contain amino acid differences as compared to the germline sequence, due to, for example, naturally-occurring somatic mutations or intentional introduction of site-directed mutation. However, a humanized antibody typically is at least 90% identical in amino acids sequence to an amino acid sequence encoded by a human germline immunoglobulin gene and contains amino acid residues that identify the antibody as being derived from human sequences when compared to the germline immunoglobulin amino acid sequences of other species (e.g., murine germline sequences). In certain cases, a humanized antibody may be at least 95, 96, 97, 98 or 99%, or even at least 96%, 97%, 98%, or 99% identical in amino acid sequence to the amino acid sequence encoded by the germline immunoglobulin gene excluding the CDRs. That is, the framework regions of the variable region (either heavy or light) can be at least 96%, 97%, 98%, or 99% identical in amino acid sequence to the framework amino acids encoded by one human germline immunoglobulin gene while the CDRs are identical to those shown in FIG. 3 (i.e., CDRs identical to those for CPA.9.086.H4(S241P).

### B. TIGIT Antibodies

The present invention provides anti-TIGIT antibodies with CDRs identical to those shown in FIG. 3 (i.e., CDRs identical to those for CPA.9.086.H4(S241P). (For convenience, “anti-TIGIT antibodies” and “TIGIT antibodies” are used interchangeably). The anti-TIGIT antibodies with CDRs identical to those shown in FIG. 3 (i.e., CDRs identical to those for CPA.9.086.H4(S241P) of the invention specifically bind to human TIGIT, and preferably the ECD of human TIGIT. The invention further provides anti-TIGIT antigen binding domains, including full length antibodies, which contain the CDRs identical to those for CPA.9.086 (S241P) and that bind to TIGIT.

Specific binding for TIGIT or a TIGIT epitope can be exhibited, for example, by an antibody having a K_{D} of at least about 10⁻⁴ M, at least about 10⁻⁵ M, at least about 10⁻⁶ M, at least about 10⁻⁷ M, at least about 10⁻⁸ M, at least about 10⁻⁹ M, alternatively at least about 10⁻¹⁰ M, at least about 10⁻¹¹ M, at least about 10⁻¹² M, at least about 10⁻¹³ M, at least about 10⁻¹⁴ M, at least about 10⁻¹⁵ M, or greater, where K_{D} refers to the equilibrium dissociation constant of a particular antibody-antigen interaction. Typically, an antibody that specifically binds an antigen will have a K_{D} that is 20-, 50-, 100-, 500-, 1000-, 5,000-, 10,000- or more times greater for a control molecule relative to the TIGIT antigen or epitope.

However, for optimal binding to TIGIT expressed on the surface of NK and T-cells, the anti-TIGIT antibodies with CDRs identical to those shown in FIG. 3 (i.e., CDRs identical to those for CPA.9.086.H4(S241P) preferably have a KD less 50 nM and most preferably less than 1 nM, with less than 0.1 nM and less than 1 pM finding use in the methods of the invention

Also, specific binding for a particular antigen or an epitope can be exhibited, for example, by anti-TIGIT antibodies with CDRs identical to those shown in FIG. 3 (i.e., CDRs identical to those for CPA.9.086.H4(S241P) having a Ka (referring to the association rate constant) for a TIGIT antigen or epitope of at least 20-, 50-, 100-, 500-, 1000-, 5,000-, 10,000- or more times greater for the epitope relative to a control, where ka refers to the association rate constant of a particular antibody-antigen interaction.

In some embodiments, the anti-TIGIT antibodies with CDRs identical to those shown in FIG. 3 (i.e., CDRs identical to those for CPA.9.086.H4(S241P) of the invention bind to human TIGIT with a K_{D} of 100 nM or less, 50 nM or less, 10 nM or less, or 1 nM or less (that is, higher binding affinity), or 1 pM or less, wherein K_{D} is determined by known methods, e.g. surface plasmon resonance (SPR, e.g., Biacore assays), ELISA, KINEXA, and most typically SPR at 25° or 37° C.

The anti-TIGIT antibodies with CDRs identical to those shown in FIG. 3 (i.e., CDRs identical to those for CPA.9.086.H4(S241P) described herein are labeled as follows. The antibodies have reference numbers, for example “CPA.9.086”. This represents the combination of the variable heavy and variable light chains, as depicted in FIG. 3, for example, with the understanding that these antibodies include two heavy chains and two light chains. “CPA.9.086. VH” refers to the variable heavy portion of CPA. 9.086, while “CPA. 9.086. VL” is the variable light chain. “CPA. 9.086.vhCDR1”, “CPA. 9.086.vhCDR2”, “CPA. 9.086.vhCDR3”, “CPA. 9.086.vlCDR1”, “CPA. 9.086.vlCDR2”, and “CPA. 9.086.vlCDR3”, refers to the CDRs are indicated. “CPA. 9.086.HC” refers to the entire heavy chain (e.g., variable and constant domain) of this molecule, and “CPA. 9.086.LC” refers to the entire light chain (e.g., variable and constant domain) of the same molecule. In general, the human kappa light chain is used for the constant domain of each phage (or humanized hybridoma) antibody herein, although in some embodiments the lambda light constant domain is used. “CPA. 9.086.H4” would be the CPA.9.086 variable domains linked to a Human IgG4. Note that in some cases, the human IgGs may have additional mutations, such are described below, and this can be annotated. For example, in many embodiments, there may be a S241P mutation in the human IgG4, and this can be annotated as “CPA.9.086.H4(S241P)” for example. The human IgG4 sequence with this S241P hinge variant is shown in FIG. 3. The invention further provides variable heavy and light domains as well as full length heavy and light chains, as provided in FIG. 3.

In some embodiments, the invention provides scFvs that bind to TIGIT comprising a variable heavy domain and a variable light domain linked by an scFv linker as outlined above and which comprise CDRs identical to those shown in FIG. 3 (i.e., CDRs identical to those for CPA.9.086.H4(S241P). The VL and VH domains can be in either orientation, e.g., from N- to C-terminus “VH-linker-VL” or “VL-linker” or “VH”. These are named by their component parts; for example, “scFv-CPA. 9.086 VH-linker-VL” or “scFv-CPA.9.086.VL-linker-VH.” Thus, “scFv-CPA.9.086” can be in either orientation.

In many embodiments, the anti-TIGIT antibodies with CDRs identical to those shown in FIG. 3 (i.e., CDRs identical to those for CPA.9.086.H4(S241P) of the invention are human (derived from phage) and block binding of TIGIT and PVR and an exemplary antibody with their components outlined (as provided in FIG. 3):
CPA.9.086, CPA.9.086. VH, CPA.9.086.VL, CPA.9.086.HC, CPA.9.086.LC,
CPA.9.086.H1, CPA.9.086.H2, CPA.9.086.H3; CPA.9.086.H4;
CPA.9.086.H4(S241P); CPA.9.086.vhCDR1, CPA.9.086.vhCDR2,
CPA.9.086.vhCDR3, CPA.9.086.vlCDR1, CPA.9.086.vlCDR2, CPA.9.086.vlCDR3 and scFv-CPA.9.086.

In addition, the anti-TIGIT antibodies with CDRs identical to those shown in FIG. 3 (i.e., CDRs identical to those for CPA.9.086.H4(S241P) include variants of the above variable heavy and light chains. In this case, the variable heavy chains can be 80%, 90%, 95%, 98% or 99% identical to the “VH” sequences herein, and/or contain from 1, 2, 3, 4, 5, 6, 7, 8, 9, 10 amino acid changes, or more, when Fc variants are used. Variable light chains are provided that can be 80%, 90%, 95%, 98% or 99% identical to the “VL” sequences herein (and in particular CPA.9.086), and/or contain from 1, 2, 3, 4, 5, 6, 7, 8, 9, 10 amino acid changes, or more, when Fc variants are used. In these embodiments, the invention includes these variants as long as the antibody still binds to TIGIT and comprises CDRs identical to those for CPA.9.086 (S241P).

Similarly, heavy and light chains of anti-TIGIT antibodies with CDRs identical to those shown in FIG. 3 (i.e., CDRs identical to those for CPA.9.086.H4(S241P) are provided that are 80%, 90%, 95%, 98% or 99% identical to the full length “HC” and “LC” sequences, and/or contain from 1, 2, 3, 4, 5, 6, 7, 8, 9, 10 amino acid changes, or more, when Fc variants are used. In these embodiments, the invention includes these variants as long as the antibody still binds to TIGIT and comprises CDRs identical to those for CPA.9.086 (S241P). S

In addition, the framework regions of the variable heavy and variable light chains of the anti-TIGIT antibodies with CDRs identical to those shown in FIG. 3 (i.e., CDRs identical to those for CPA.9.086.H4(S241P) herein can be humanized as is known in the art (with occasional variants generated in the CDRs as needed), and thus humanized variants of the VH and VL chains of FIG. 3 can be generated. Furthermore, the humanized variable heavy and light domains can then be fused with human constant regions, such as the constant regions from IgG4 (including IgG4 (S241P)).

In particular, as is known in the art, murine VH and VL chains can be humanized as is known in the art, for example, using the IgBLAST program of the NCBI website, as outlined in Ye et al. Nucleic Acids Res. 41: W34-W40 (2013), herein incorporated by reference in its entirety for the humanization methods. IgBLAST takes a murine VH and/or VL sequence and compares it to a library of known human germline sequences.

Accordingly, the anti-TIGIT antibodies with CDRs identical to those shown in FIG. 3 (i.e., CDRs identical to those for CPA.9.086.H4(S241P) of the invention comprise amino acid sequences selected from the group consisting of (a) sequences as listed herein; (b) amino acid sequences having 90% or greater, 95% or greater, 98% or greater, or 99% or greater sequence identity to the sequences specified in (a) and with CDRs identical to those shown in FIG. 3 (i.e., CDRs identical to those for CPA.9.086.H4(S241P); (c) a polypeptide having an amino acid sequence encoded by a polynucleotide having a nucleic acid sequence encoding the amino acids as listed herein. In particular, the CPA.9.086 antibody can have sequences selected from (a), (b), or (c).

Additionally, included in the definition of the anti-TIGIT antibodies with CDRs identical to those shown in FIG. 3 (i.e., CDRs identical to those for CPA.9.086.H4(S241P) are antibodies that share identity to the TIGIT antibodies enumerated herein but maintain the CDRs identical to those shown in FIG. 3 (i.e., CDRs identical to those for CPA.9.086.H4(S241P). That is, in certain embodiments, an anti-TIGIT antibody according to the invention comprises heavy and light chain variable regions comprising amino acid sequences that are identical to all or part of the anti-TIGIT amino acid sequences, respectively, wherein the antibodies retain the desired functional properties of the parent anti-TIGIT antibodies and comprise CDRs identical to those shown in FIG. 3 (i.e., CDRs identical to those for CPA.9.086.H4(S241P). The percent identity between the two sequences is a function of the number of identical positions shared by the sequences (i.e., % homology=# of identical positions/total # of positions ×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package (available commercially), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present invention can further be used as a “query sequence” to perform a search against public databases to, for example, identify related sequences. Such searches can be performed using the XBLAST program (version 2.0) of Altschul, et al. (1990) J Mol. Biol. 215:403-10. BLAST protein searches can be performed with the XBLAST program, score=50, wordlength=3 to obtain amino acid sequences homologous to the antibody molecules according to at least some embodiments of the invention. To obtain gapped alignments for comparison purposes, Gapped BLAST can be utilized as described in Altschul et al., (1997) Nucleic Acids Res. 25 (17): 3389-3402. When utilizing BLAST and Gapped BLAST programs, the default parameters of the respective programs (e.g., XBLAST and NBLAST) can be used.

In general, the percentage identity for comparison between anti-TIGIT antibodies with CDRs identical to those shown in FIG. 3 (i.e., CDRs identical to those for CPA.9.086.H4(S241P) is at least 75%, at least 80%, at least 90%, with at least about 95, 96, 97, 98 or 99% percent identity being preferred. The percentage identity may be along the whole amino acid sequence, for example the entire heavy or light chain or along a portion of the chains, so long as the CDRs identical to those shown in FIG. 3 (i.e., CDRs identical to those for CPA.9.086.H4(S241P). For example, included within the definition of the anti-TIGIT antibodies of the invention are those that share identity along the entire variable region (for example, where the identity is 95 or 98% identical along the variable regions), or along the entire constant region, or along just the Fc domain, so long as the CDRs identical to those shown in FIG. 3 (i.e., CDRs identical to those for CPA.9.086.H4(S241P). In particular, the invention provides TIGIT antibodies that have at least 75%, at least 80%, at least 90%, with at least about 95, 96, 97, 98 or 99% percent identity being preferred, with the CPA.9.086 antibody and so long as the CDRs identical to those shown in FIG. 3 (i.e., CDRs identical to those for CPA.9.086.H4(S241P).

Also included are sequences where the TIGIT antibodies include those with CDRs identical to those shown in FIG. 3 (i.e., CDRs identical to those for CPA.9.086.H4(S241P) but whose identity along the variable region can be lower, for example 95 or 98% percent identical. In particular, the invention provides TIGIT antibodies that have identical CDRs to CPA.9.086 but with framework regions that are 95 or 98% identical to CPA.9.086.

### IV. Formulations of Anti-Tigit Antibodies

The anti-TIGIT antibodies with CDRs identical to those shown in FIG. 3 (i.e., CDRs identical to those for CPA.9.086.H4(S241P) can be formulated into pharmaceutical compositions comprising a carrier suitable for the desired delivery method. Suitable carriers include any material that when combined with the therapeutic composition retains the anti-tumor function of the therapeutic composition and is generally non-reactive with the patient's immune system. Examples include, but are not limited to, any of a number of standard pharmaceutical carriers such as sterile phosphate buffered saline solutions, bacteriostatic water, and the like (see, generally, Remington's Pharmaceutical Sciences 16^{th} Edition, A. Osal., Ed., 1980).

As used herein, the term “activity” refers to a functional activity or activities of anti-TIGIT antibodies with CDRs identical to those shown in FIG. 3 (i.e., CDRs identical to those for CPA.9.086.H4(S241P) and/or antigen binding portions thereof, so long as they comprise CDRs identical to those for CPA.9.086 (S241P). Functional activities include, but are not limited to, biological activity and/or binding affinity.

As used herein, the term “stability” is used in a structural context, e.g., relating to the structural integrity of anti-TIGIT antibodies with CDRs identical to those shown in FIG. 3 (i.e., CDRs identical to those for CPA.9.086.H4(S241P) and/or antigen binding portion thereof, or in a functional context, e.g., relating to anti-TIGIT antibodies with CDRs identical to those shown in FIG. 3 (i.e., CDRs identical to those for CPA.9.086.H4(S241P) and/or antigen binding portion thereof's ability to retain its function and/or activity over time. As will be appreciated, the anti-TIGIT antibodies with CDRs identical to those shown in FIG. 3 (i.e., CDRs identical to those for CPA.9.086.H4(S241P) may be contained within a formulation in accordance with the methods and compositions described herein, and the stability of that protein refers to its stability in that formulation. In some embodiments, the stability of an anti-TIGIT antibodies with CDRs identical to those shown in FIG. 3 (i.e., CDRs identical to those for CPA.9.086.H4(S241P) comprising composition is determined by measuring the binding activity of the composition, including for example, using the assays described in the application and figures provided herewith, as well as other applicable assays known in the art.

As used herein, a “storage stable” aqueous anti-TIGIT antibodies with CDRs identical to those shown in FIG. 3 (i.e., CDRs identical to those for CPA.9.086.H4(S241P) comprising composition refers to solution that has been formulated to increase the stability of the protein in solution, for example by at least 10%, over a given storage time.

As used herein, “shelf-life” refers to the period of time a formulation maintains a predetermined level of stability at a predetermined temperature. In particular embodiments, the predetermined temperature refers to frozen (e.g., −80° C., −25° C., 0° C.), refrigerated (e.g., 0° to 10° C.), or room temperature (e.g., 18° C. to 32° C.) storage.

As used herein, the term “time of stability” refers to the length of time a formulation is considered stable. For example, the time of stability for a formulation may refer to the length of time for which the level of protein aggregation and/or degradation in the formulation remains below a certain threshold (e.g., 1%, 2%, 3%, 4%, 5%, 6%, 7%, 8%, 9%, 10%, 11%, 12%, 13%, 14%, 15%, 16%, 17%, 18%, 19%, 20%, etc.), and/or the length of time a formulation maintains biological activity above a certain threshold (e.g., 100%, 95%, 90%, 85%, 80%, 75%, 70%, 65%, 60%, 55%, 50%, etc.) of the amount of activity (including, for example, binding activity) present in the formulation at the start of the storage period.

### A. Stabilized Liquid Formulations

In some embodiments, the present disclosure provides stabilized aqueous formulations of anti-TIGIT antibodies with CDRs identical to those shown in FIG. 3 (i.e., CDRs identical to those for CPA.9.086.H4(S241P). The following embodiments are based in part on the discovery that inclusion of at least one amino acid, salt, and/or non-ionic surfactant stabilizes the liquid anti-TIGIT antibodies with CDRs identical to those shown in FIG. 3 (i.e., CDRs identical to those for CPA.9.086.H4(S241P) compositions, as compared to compositions lacking the at least one amino acid, salt, and/or non-ionic surfactant. In some embodiments, the formulation does not comprise a sugar and/or sugar alcohol.

In one embodiment, a storage stable anti-TIGIT antibody with CDRs identical to those shown in FIG. 3 (i.e., CDRs identical to those for CPA.9.086.H4(S241P) provided herein will be stabilized at a temperature between 5° C. and 25° C.) for a period of time. In some embodiments, the anti-TIGIT antibodies with CDRs identical to those shown in FIG. 3 (i.e., CDRs identical to those for CPA.9.086.H4(S241P) comprising composition will be stable at refrigerated temperature for at least 1 day, 2 days, 3 days, 4 days, 5 days, 6 days, 7 days, 8 days, 9 days, 10 days, 11 days, 12 days, 13 days, 14 days, 21 days, 28 days, or more days. In some embodiments, the anti-TIGIT antibodies with CDRs identical to those shown in FIG. 3 (i.e., CDRs identical to those for CPA.9.086.H4(S241P) comprising composition will be stable for at least 1 week, 2 weeks, 3 weeks, 4 weeks, 5 weeks, 6 weeks, 7 weeks, 8 weeks, 9 weeks, 10 weeks, or more. In some embodiments, the anti-TIGIT antibodies with CDRs identical to those shown in FIG. 3 (i.e., CDRs identical to those for CPA.9.086.H4(S241P) comprising composition will be stable for at least 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, or more months. In some embodiments, the anti-TIGIT antibodies with CDRs identical to those shown in FIG. 3 (i.e., CDRs identical to those for CPA.9.086.H4(S241P) comprising composition will be stable for an extended period of time when stored at a temperature between 5° C. and 25° C. In some embodiments, the anti-TIGIT antibodies with CDRs identical to those shown in FIG. 3 (i.e., CDRs identical to those for CPA.9.086.H4(S241P) comprising composition will be stable for an extended period of time when stored at a temperature between 10° C. and 25° C. In some embodiments, the anti-TIGIT antibodies with CDRs identical to those shown in FIG. 3 (i.e., CDRs identical to those for CPA.9.086.H4(S241P) comprising composition will be stable for an extended period of time when stored at a temperature between 15° C. and 25° C. In some embodiments, the anti-TIGIT antibodies with CDRs identical to those shown in FIG. 3 (i.e., CDRs identical to those for CPA.9.086.H4(S241P) comprising composition will be stable for an extended period of time when stored at a temperature between 15° C. and 20° C. In some embodiments, the anti-TIGIT antibodies with CDRs identical to those shown in FIG. 3 (i.e., CDRs identical to those for CPA.9.086.H4(S241P) composition will be stable for an extended period of time when stored at a temperature between 20° C. and 25° C. In some embodiments, the anti-TIGIT antibodies with CDRs identical to those shown in FIG. 3 (i.e., CDRs identical to those for CPA.9.086.H4(S241P) composition will be stable for an extended period of time when stored at a temperature of about 25° C.

In one embodiment, a stored anti-TIGIT antibodies with CDRs identical to those shown in FIG. 3 (i.e., CDRs identical to those for CPA.9.086.H4(S241P) composition is considered storage stable as long as the composition maintains at least 40% of the antibody binding activity present at the start of the storage period (e.g., at time=0). In another embodiment, a stored composition is considered stable as long as the composition maintains at least 45%, 50%, 55%, 60%, 65%, 70%, 75%, 80%, 85%, 90%, 95% or more of the antibody binding activity present at the start of the storage period (e.g., at time=0). In one embodiment, antibody binding activity is measure using any assay known in the art.

In one embodiment, a stored anti-TIGIT antibody with CDRs identical to those shown in FIG. 3 (i.e., CDRs identical to those for CPA.9.086.H4(S241P) composition is considered stable as long as the percentage of anti-TIGIT antibodies with CDRs identical to those shown in FIG. 3 (i.e., CDRs identical to those for CPA.9.086.H4(S241P) present in an aggregated state remains no more than 50%. In some embodiments, a stored anti-TIGIT antibodies with CDRs identical to those shown in FIG. 3 (i.e., CDRs identical to those for CPA.9.086.H4(S241P) composition is considered stable as long as the percentage of the anti-TIGIT antibodies with CDRs identical to those shown in FIG. 3 (i.e., CDRs identical to those for CPA.9.086.H4(S241P) thereof present in an aggregated state remains no more than 45%, 40%, 35%, 30%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, or less.

In some embodiments, a stored anti-TIGIT antibodies with CDRs identical to those shown in FIG. 3 (i.e., CDRs identical to those for CPA.9.086.H4(S241P) composition is considered stable as long as the composition maintains at least 40% of the starting binding activity (e.g., at time=0) after being subjected to mechanical stress. In another embodiment, a stored anti-TIGIT antibodies with CDRs identical to those shown in FIG. 3 (i.e., CDRs identical to those for CPA.9.086.H4(S241P) composition is considered stable as long as the composition maintains 45%, 50%, 55%, 60%, 65%, 70%, 75%, 80%, 85%, 90%, 95% or more of the starting binding activity (e.g., at time=0) after being subjected to mechanical stress. In some embodiments, the mechanical stress is agitation (e.g., shaking).

In some embodiments, shelf life is determined by a percent activity remaining after storage at any of the above temperatures for any of the above periods of time. In some embodiments, shelf life means that the formulation retains at least 10%, 15%, 20%, 25%, 30%, 35%, 40%, 45%, 50%, 55%, 60%, 65%, 70%, 75%, 80%, 85%, 90%, 95%, 96%, 97%, 98%, 99%, 100% of antibody activity as measured by any of the assays described herein or known in the art as compared to activity prior to storage for any of the above amounts of time at any of the above temperatures.

In some embodiments, the anti-TIGIT antibodies with CDRs identical to those shown in FIG. 3 (i.e., CDRs identical to those for CPA.9.086.H4(S241P) is at a concentration of from 10 mg/mL to 25 mg/mL, 10 mg/mL to 15 mg/mL, 10 mg/mL to 20 mg/mL, 15 mg/mL to 20 mg/mL, 15 mg/mL to 25 mg/mL, or 20 mg/mL to 25 mg/mL. In some embodiments, the anti-TIGIT antibodies with CDRs identical to those shown in FIG. 3 (i.e., CDRs identical to those for CPA.9.086.H4(S241P) is at a concentration of from 10 mg/mL to 25 mg/mL. In some embodiments, the anti-TIGIT antibodies with CDRs identical to those shown in FIG. 3 (i.e., CDRs identical to those for CPA.9.086.H4(S241P) is at a concentration of from 10 mg/mL to 15 mg/mL. In some embodiments, the anti-TIGIT antibodies with CDRs identical to those shown in FIG. 3 (i.e., CDRs identical to those for CPA.9.086.H4(S241P) is at a concentration of from 10 mg/mL to 20 mg/mL. In some embodiments, the anti-TIGIT antibodies with CDRs identical to those shown in FIG. 3 (i.e., CDRs identical to those for CPA.9.086.H4(S241P) is at a concentration of from 10 mg/mL to 25 mg/mL. In some embodiments, the anti-TIGIT antibodies with CDRs identical to those shown in FIG. 3 (i.e., CDRs identical to those for CPA.9.086.H4(S241P) is at a concentration of from 15 mg/mL to 20 mg/mL. In some embodiments, the anti-TIGIT antibodies with CDRs identical to those shown in FIG. 3 (i.e., CDRs identical to those for CPA.9.086.H4(S241P) is at a concentration of from 15 mg/mL to 25 mg/mL. In some embodiments, the anti-TIGIT antibodies with CDRs identical to those shown in FIG. 3 (i.e., CDRs identical to those for CPA.9.086.H4(S241P) is at a concentration of 15 mg/mL. In some embodiments, the anti-TIGIT antibodies with CDRs identical to those shown in FIG. 3 (i.e., CDRs identical to those for CPA.9.086.H4(S241P) is at a concentration of 20 mg/mL. In some embodiments, the anti-TIGIT antibodies with CDRs identical to those shown in FIG. 3 (i.e., CDRs identical to those for CPA.9.086.H4(S241P) is at a concentration of 25 mg/mL.

### B. Amino Acids-Histidine and Arginine

In some embodiments, the present invention provides a stable liquid pharmaceutical formulation of anti-TIGIT antibodies with CDRs identical to those shown in FIG. 3 (i.e., CDRs identical to those for CPA.9.086.H4(S241P) comprising 8 mM to 12 mM histidine, from 8 mM to 10 mM histidine, from 10 mM to 612 mM histidine, or about 10 mM histidine. In some embodiments, the pharmaceutical formulation comprises from 8 mM to 12 mM histidine. In some embodiments, the pharmaceutical formulation comprises from 8 mM to 10 mM histidine. In some embodiments, the pharmaceutical formulation comprises from 10 mM to 12 mM histidine. In some embodiments, the pharmaceutical formulation comprises about 10 mM histidine.

In some embodiments, the present invention provides a stable liquid pharmaceutical formulation of anti-TIGIT antibodies with CDRs identical to those shown in FIG. 3 (i.e., CDRs identical to those for CPA.9.086.H4(S241P) comprising from 25 mM to 35 mM L-arginine, from 25 mM to 30 mM L-arginine, from 30 mM to 35 mM L-arginine, about 20 mM L-arginine, about 25 mM L-arginine, about 30 mM L-arginine, or about 35 mM L-arginine. In some embodiments, the present invention provides a stable liquid pharmaceutical formulation of anti-TIGIT antibodies with CDRs identical to those shown in FIG. 3 (i.e., CDRs identical to those for CPA.9.086.H4(S241P) comprising from 25 mM to 35 mM L-arginine. In some embodiments, the present invention provides a stable liquid pharmaceutical formulation of anti-TIGIT antibodies with CDRs identical to those shown in FIG. 3 (i.e., CDRs identical to those for CPA.9.086.H4(S241P) comprising from 25 mM to 30 mM L-arginine. In some embodiments, the present invention provides a stable liquid pharmaceutical formulation of anti-TIGIT antibodies with CDRs identical to those shown in FIG. 3 (i.e., CDRs identical to those for CPA.9.086.H4(S241P) comprising from 30 mM to 35 mM L-arginine. In some embodiments, the present invention provides a stable liquid pharmaceutical formulation of anti-TIGIT antibodies with CDRs identical to those shown in FIG. 3 (i.e., CDRs identical to those for CPA.9.086.H4(S241P) comprising about 20 mM L-arginine. In some embodiments, the present invention provides a stable liquid pharmaceutical formulation of anti-TIGIT antibodies with CDRs identical to those shown in FIG. 3 (i.e., CDRs identical to those for CPA.9.086.H4(S241P) comprising about 25 mM L-arginine. In some embodiments, the present invention provides a stable liquid pharmaceutical formulation of anti-TIGIT antibodies with CDRs identical to those shown in FIG. 3 (i.e., CDRs identical to those for CPA.9.086.H4(S241P) comprising about 30 mM L-arginine. In some embodiments, the present invention provides a stable liquid pharmaceutical formulation of anti-TIGIT antibodies with CDRs identical to those shown in FIG. 3 (i.e., CDRs identical to those for CPA.9.086.H4(S241P) comprising about 35 mM L-arginine.

### C. Sucrose

In some embodiments, the present invention provides a stable liquid pharmaceutical formulation of anti-TIGIT antibodies with CDRs identical to those shown in FIG. 3 (i.e., CDRs identical to those for CPA.9.086.H4(S241P) comprising no sugar alcohol.

In some embodiments, the present invention provides a stable liquid pharmaceutical formulation of anti-TIGIT antibodies with CDRs identical to those shown in FIG. 3 (i.e., CDRs identical to those for CPA.9.086.H4(S241P) comprising from about 7.0% to 9.0%, 7.5% to 8.5%, 7.5% to 9.0%, 8.0% to 8.5%, 8.0% to 9%, or 8.5% to 9% sucrose. In some embodiments, the present invention provides a stable liquid pharmaceutical formulation of anti-TIGIT antibodies with CDRs identical to those shown in FIG. 3 (i.e., CDRs identical to those for CPA.9.086.H4(S241P) comprising about 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0% sucrose. In some embodiments, the present invention provides a stable liquid pharmaceutical formulation of anti-TIGIT antibodies with CDRs identical to those shown in FIG. 3 (i.e., CDRs identical to those for CPA.9.086.H4(S241P) comprising about 7.5%, 7.6%, 7.7%, 7.8%, 7.9%, 8.0%, 8.1%, 8.2%, 8.3%, or 8.4% sucrose. In some embodiments, the present invention provides a stable liquid pharmaceutical formulation of anti-TIGIT antibodies with CDRs identical to those shown in FIG. 3 (i.e., CDRs identical to those for CPA.9.086.H4(S241P) comprising about 7.9%, 8.0%, 8.1%, or 8.2% sucrose. In some embodiments, the present invention provides a stable liquid pharmaceutical formulation of anti-TIGIT antibodies with CDRs identical to those shown in FIG. 3 (i.e., CDRs identical to those for CPA.9.086.H4(S241P) comprising about 8.0% sucrose.

### D. Polysorbate 80

In some embodiments, the present invention provides a stable liquid pharmaceutical formulation of anti-TIGIT antibodies with CDRs identical to those shown in FIG. 3 (i.e., CDRs identical to those for CPA.9.086.H4(S241P) comprising the non-ionic surfactant polysorbate 80 (also known as polyoxyethylene (20) sorbitan monooleate) in a range of 70 ppm to 80 ppm.

In some embodiments, the present invention provides a stable liquid pharmaceutical formulation of anti-TIGIT antibodies with CDRs identical to those shown in FIG. 3 (i.e., CDRs identical to those for CPA.9.086.H4(S241P) comprising the non-ionic surfactant polysorbate 80 (also known as polyoxyethylene (20) sorbitan monooleate) at about 70 ppm, about 75 ppm, or about 80 ppm.

### E. pH

In some embodiments, the present invention provides a stable liquid pharmaceutical formulation of anti-TIGIT antibodies with CDRs identical to those shown in FIG. 3 (i.e., CDRs identical to those for CPA.9.086.H4(S241P) that is buffered at a physiologically acceptable pH of from about 5.2 to about 5.6. In some embodiments, the pH is about 5.2, 5.3, 5.4, 5.5, or 5.6. In some embodiments, the pH is from 5.2 to 5.5. In some embodiments, the pH is from 5.3 to 5.5. In some embodiments, the pH is from 5.4 to 5.5. In some embodiments, the pH is from 5.3 to 5.6. In some embodiments, the pH is from 5.4 to 5.6. In some embodiments, the pH is from 5.5 to 5.6. In some embodiments, the pH is 5.4+/−0.2.

### F. Stability Assays

As discussed herein, the stable liquid pharmaceutical formulations comprising anti-TIGIT antibodies with CDRs identical to those shown in FIG. 3 (i.e., CDRs identical to those for CPA.9.086.H4(S241P) show improved stability as compared to control formulations. In one embodiment, improved stability includes retention of a higher percentage of binding activity and/or no reduction in binding activity as compared to control formulations in various stability assays. Such assays can be used to determine if a formulation is a highly stabilized formulation. In some embodiments, the highly stabilized formulation has at least 5%, 10%, 20%, 30%, 40%, 50%, 55%, 60%, 65%, 70%, 75%, 80%, 85%, 90%, 95%, 96%, 97%, 98%, 99% or greater activity than a control formulation when assessed by any of the stability assays discussed herein or known in the art.

In some embodiments, the liquid pharmaceutical formulations comprising anti-TIGIT antibodies with CDRs identical to those shown in FIG. 3 (i.e., CDRs identical to those for CPA.9.086.H4(S241P) thereof are tested under stressor conditions, such as storage at high temperature, agitation, freeze/thaw cycles, or some combination thereof. After such stressors, the formulations are assayed using any of the methods described herein or known in the art to determine the stability under these conditions.

In some embodiments, an A280 by SoloVPE assay can be employed to examine the appearance of the stable liquid pharmaceutical formulations comprising anti-TIGIT antibodies with CDRs identical to those shown in FIG. 3 (i.e., CDRs identical to those for CPA.9.086.H4(S241P). In some embodiments, the SoloVPE assay can be employed to examine concentrations for the stable liquid pharmaceutical formulations comprising anti-TIGIT antibodies with CDRs identical to those shown in FIG. 3 (i.e., CDRs identical to those for CPA.9.086.H4(S241P). A280: Amino acids containing aromatic side chains exhibit strong UV-light absorption at the wavelength of 280 nm. Once an absorptivity coefficient has been established for a given protein, the protein's concentration in solution can be calculated from its absorbance. The method is designed to determine the protein concentration by measuring its absorbance at 280 nm using the SoloVPE instrument without dilution (https://www.ctechnologiesinc.com/products/solovpe).

In some embodiments, a binding assay can be performed to examine the activity of the stable liquid pharmaceutical formulations comprising anti-TIGIT antibodies with CDRs identical to those shown in FIG. 3 (i.e., CDRs identical to those for CPA.9.086.H4(S241P).

### LabChip Analysis

In some embodiments, a LabChip analysis is employed to examine purity, including for example, IgG purity as well as HC+LC percentages for the stable liquid pharmaceutical formulations comprising anti-TIGIT antibodies with CDRs identical to those shown in FIG. 3 (i.e., CDRs identical to those for CPA.9.086.H4(S241P). In some embodiments, the stable liquid pharmaceutical formulations comprising anti-TIGIT antibodies with CDRs identical to those shown in FIG. 3 (i.e., CDRs identical to those for CPA.9.086.H4(S241P) exhibit IgG purity percentages greater than 94%, greater than 95%, greater than 96%, greater than 97%, or greater than 98%. In some embodiments, the stable liquid pharmaceutical formulations comprising anti-TIGIT antibodies with CDRs identical to those shown in FIG. 3 (i.e., CDRs identical to those for CPA.9.086.H4(S241P) exhibit IgG purity percentages were from about 95% to 98%. In some embodiments, the stable liquid pharmaceutical formulations comprising anti-TIGIT antibodies with CDRs identical to those shown in FIG. 3 (i.e., CDRs identical to those for CPA.9.086.H4(S241P) exhibit IgG purity percentages from about 96% to 97%. In some embodiments, the stable liquid pharmaceutical formulations comprising anti-TIGIT antibodies with CDRs identical to those shown in FIG. 3 (i.e., CDRs identical to those for CPA.9.086.H4(S241P) exhibit HC+LC percentages from about 96% to 100%. In some embodiments, the stable liquid pharmaceutical formulations comprising anti-TIGIT antibodies with CDRs identical to those shown in FIG. 3 (i.e., CDRs identical to those for CPA.9.086.H4(S241P) exhibit HC+LC percentages from about 97% to 100%. In some embodiments, the stable liquid pharmaceutical formulations comprising anti-TIGIT antibodies with CDRs identical to those shown in FIG. 3 (i.e., CDRs identical to those for CPA.9.086.H4(S241P) exhibit HC+LC percentages from about 98% to 100%.

In some embodiments, a capillary isoelectric focusing (cIEF) can be employed to analyze the stable liquid pharmaceutical formulations comprising a anti-TIGIT antibodies with CDRs identical to those shown in FIG. 3 (i.e., CDRs identical to those for CPA.9.086.H4(S241P) for the presence of additional species, including for example, minor acidic species.

Antibodies can form sub-visible particles in response to stressed conditions, such as heat, freeze/thaw cycles, and agitation. In some embodiments, a microflow imaging (MFI) analysis can be employed to analyze the stable liquid pharmaceutical formulations comprising anti-TIGIT antibodies with CDRs identical to those shown in FIG. 3 (i.e., CDRs identical to those for CPA.9.086.H4(S241P) for the formation of particles in response to stressed conditions. In some embodiments, the stable liquid pharmaceutical formulations of the anti-TIGIT antibodies with CDRs identical to those shown in FIG. 3 (i.e., CDRs identical to those for CPA.9.086.H4(S241P) provide for a formulation capable of stabilizing against these stressed conditions and protecting against the formation of particles. MFI can be used to evaluate particle counts at different size ranges (<2 μm, <5 μm, <10 μm, and <25 μm) in different formulations under stressed conditions. Typically, MFI data can be evaluated to choose an appropriate formulation based on generation of the lowest amount of particles/mL for all sizes of particles across all time points, conditions, and formulations.

In some embodiments, size exclusion chromatography (SEC) can be employed to analyze the stable liquid pharmaceutical formulations comprising the anti-TIGIT antibodies with CDRs identical to those shown in FIG. 3 (i.e., CDRs identical to those for CPA.9.086.H4(S241P).

### G. Selected Formulation Embodiments

In some embodiments, the present invention provides a stable liquid pharmaceutical formulation of an anti-TIGIT antibody comprising:
(a) an anti-TIGIT antibody, the anti-TIGIT antibody comprising:
   i) a heavy chain variable domain comprising the vhCDR1, vhCDR2, and vhCDR3 from the heavy chain of CPA.9.086.H4(S241P) (SEQ ID NO:5); and
   ii) ii) a light chain variable domain comprising the vlCDR1, vlCDR2, and vlCDR3 from the light chain of CPA.9.086.H4(S241P) (SEQ ID NO:10);
(b) from 8 mM to 12 mM histidine;
(c) from 25 mM to 35 mM L-Arginine;
(d) from 8 mM to 12 mM L-methionine;
(e) from 7% (w/v) to 9% (w/v) sucrose; and
(f) from 70 ppm to 80 ppm polysorbate 80,
wherein the pharmaceutical formulation has a pH of 5.4+/−0.2.

In some embodiments, the present invention provides a stable liquid pharmaceutical formulation of an anti-TIGIT antibody comprising:
(a) about 20 mg/mL of an anti-TIGIT antibody, the anti-TIGIT antibody comprising:
   i) a heavy chain variable domain comprising the vhCDR1, vhCDR2, and vhCDR3 from the heavy chain of CPA.9.086.H4(S241P) (SEQ ID NO:5); and
   ii) a light chain variable domain comprising the vlCDR1, vlCDR2, and vlCDR3 from the light chain of CPA.9.086.H4(S241P) (SEQ ID NO:10);
(b) about 10 mM histidine;
(c) about 30 mM L-Arginine;
(d) about 10 mM L-methionine;
(e) about 8% (w/v) sucrose; and
(f) about 75 ppm polysorbate 80,
wherein the pharmaceutical formulation has a pH of 5.4+/−0.2.

In some embodiments, the present invention provides a stable liquid pharmaceutical formulation comprising:
(a) about 20 mg/mL of an anti-TIGIT antibody, the anti-TIGIT antibody comprising:
   i) a heavy chain variable domain from the heavy chain of CPA.9.086.H4(S241P) (SEQ ID NO:1); and
   ii) a light chain variable domain from the light chain of CPA.9.086.H4(S241P) (SEQ ID NO:6);
(b) about 10 mM histidine;
(c) about 30 mM L-Arginine;
(d) about 10 mM L-methionine;
(e) about 8% (w/v) sucrose; and
(f) about 75 ppm polysorbate 80,
wherein the pharmaceutical formulation has a pH of 5.4+/−0.2.

In some embodiments, the present invention provides a stable liquid pharmaceutical formulation comprising:
(a) about 20 mg/mL of an anti-TIGIT antibody, the anti-TIGIT antibody comprising:
   i) a heavy chain from CPA.9.086.H4(S241P) (SEQ ID NO:5); and
   ii) a light chain from the light chain of CPA.9.086.H4(S241P) (SEQ ID NO: 10);
(b) about 10 mM histidine;
(c) about 30 mM L-Arginine;
(d) about 10 mM L-methionine;
(e) about 8% (w/v) sucrose; and
(f) about 75 ppm polysorbate 80,
wherein the pharmaceutical formulation has a pH of 5.4+/−0.2.

### V. Administration of Anti-Tigit Antibodies

Administration of the pharmaceutical composition comprising anti-TIGIT antibodies with CDRs identical to those shown in FIG. 3 (i.e., CDRs identical to those for CPA.9.086.H4(S241P), preferably in the form of a sterile aqueous solution, may be done in a variety of ways, As is known in the art, protein therapeutics are often delivered by IV infusion. The antibodies of the present invention may also be delivered using such methods. For example, administration may venious be by intravenous infusion with 0.9% sodium chloride as an infusion vehicle. Such techniques are disclosed in Remington's Pharmaceutical Sciences 16th edition, Osol, A. Ed., 1980.

The dosing amounts and frequencies of administration are, in some embodiments, selected to be therapeutically or prophylactically effective. As is known in the art, adjustments for protein degradation, systemic versus localized delivery, and rate of new protease synthesis, as well as the age, body weight, general health, sex, diet, time of administration, drug interaction and the severity of the condition may be necessary, and will be ascertainable with routine experimentation by those skilled in the art. In order to treat a patient, a therapeutically effective dose of the Fc variant of the present invention may be administered. By “therapeutically effective dose” herein is meant a dose that produces the effects for which it is administered.

### VI. Dosing

In some embodiments, the anti-TIGIT antibodies with CDRs identical to those shown in FIG. 3 (i.e., CDRs identical to those for CPA.9.086.H4(S241P) formulations of the present invention can be formulated for administration, including as a unit dosage formulation. In some embodiments, the anti-TIGIT antibodies with CDRs identical to those shown in FIG. 3 (i.e., CDRs identical to those for CPA.9.086.H4(S241P) formulations are administered at a dosage of 0.01 mg/kg of the anti-TIGIT antibodies with CDRs identical to those shown in FIG. 3 (i.e., CDRs identical to those for CPA.9.086.H4(S241P). In some embodiments, the anti-TIGIT antibodies with CDRs identical to those shown in FIG. 3 (i.e., CDRs identical to those for CPA.9.086.H4(S241P) are administered at a dosage of 0.02 mg/kg of the anti-TIGIT antibodies with CDRs identical to those shown in FIG. 3 (i.e., CDRs identical to those for CPA.9.086.H4(S241P). In some embodiments, the anti-TIGIT antibodies with CDRs identical to those shown in FIG. 3 (i.e., CDRs identical to those for CPA.9.086.H4(S241P) are administered at a dosage of 0.03 mg/kg of the anti-TIGIT antibodies with CDRs identical to those shown in FIG. 3 (i.e., CDRs identical to those for CPA.9.086.H4(S241P). In some embodiments, the anti-TIGIT antibodies with CDRs identical to those shown in FIG. 3 (i.e., CDRs identical to those for CPA.9.086.H4(S241P) are administered at a dosage of 0.04 mg/kg of the anti-TIGIT antibodies with CDRs identical to those shown in FIG. 3 (i.e., CDRs identical to those for CPA.9.086.H4(S241P). In some embodiments, the anti-TIGIT antibodies with CDRs identical to those shown in FIG. 3 (i.e., CDRs identical to those for CPA.9.086.H4(S241P) are administered at a dosage of 0.05 mg/kg of the anti-TIGIT antibodies with CDRs identical to those shown in FIG. 3 (i.e., CDRs identical to those for CPA.9.086.H4(S241P). In some embodiments, the anti-TIGIT antibodies with CDRs identical to those shown in FIG. 3 (i.e., CDRs identical to those for CPA.9.086.H4(S241P) are administered at a dosage of 0.06 mg/kg of the anti-TIGIT antibodies with CDRs identical to those shown in FIG. 3 (i.e., CDRs identical to those for CPA.9.086.H4(S241P). In some embodiments, the anti-TIGIT antibodies with CDRs identical to those shown in FIG. 3 (i.e., CDRs identical to those for CPA.9.086.H4(S241P) are administered at a dosage of 0.07 mg/kg of the anti-TIGIT antibodies with CDRs identical to those shown in FIG. 3 (i.e., CDRs identical to those for CPA.9.086.H4(S241P). In some embodiments, the anti-TIGIT antibodies with CDRs identical to those shown in FIG. 3 (i.e., CDRs identical to those for CPA.9.086.H4(S241P) are administered at a dosage of 0.08 mg/kg of the anti-TIGIT antibodies with CDRs identical to those shown in FIG. 3 (i.e., CDRs identical to those for CPA.9.086.H4(S241P). In some embodiments, the anti-TIGIT antibodies with CDRs identical to those shown in FIG. 3 (i.e., CDRs identical to those for CPA.9.086.H4(S241P) are administered at a dosage of 0.09 mg/kg of the anti-TIGIT antibodies with CDRs identical to those shown in FIG. 3 (i.e., CDRs identical to those for CPA.9.086.H4(S241P). In some embodiments, the anti-TIGIT antibodies with CDRs identical to those shown in FIG. 3 (i.e., CDRs identical to those for CPA.9.086.H4(S241P) are administered at a dosage of 0.1 mg/kg of the anti-TIGIT antibodies with CDRs identical to those shown in FIG. 3 (i.e., CDRs identical to those for CPA.9.086.H4(S241P). In some embodiments, the anti-TIGIT antibodies with CDRs identical to those shown in FIG. 3 (i.e., CDRs identical to those for CPA.9.086.H4(S241P) are administered at a dosage of 0.2 mg/kg of the anti-TIGIT antibodies with CDRs identical to those shown in FIG. 3 (i.e., CDRs identical to those for CPA.9.086.H4(S241P). In some embodiments, the anti-TIGIT antibodies with CDRs identical to those shown in FIG. 3 (i.e., CDRs identical to those for CPA.9.086.H4(S241P) are administered at a dosage of 0.3 mg/kg of the anti-TIGIT antibodies with CDRs identical to those shown in FIG. 3 (i.e., CDRs identical to those for CPA.9.086.H4(S241P). In some embodiments, the anti-TIGIT antibodies with CDRs identical to those shown in FIG. 3 (i.e., CDRs identical to those for CPA.9.086.H4(S241P) are administered at a dosage of 0.5 mg/kg of the anti-TIGIT antibodies with CDRs identical to those shown in FIG. 3 (i.e., CDRs identical to those for CPA.9.086.H4(S241P). In some embodiments, the anti-TIGIT antibodies with CDRs identical to those shown in FIG. 3 (i.e., CDRs identical to those for CPA.9.086.H4(S241P) are administered at a dosage of 0.8 mg/kg of the anti-TIGIT antibodies with CDRs identical to those shown in FIG. 3 (i.e., CDRs identical to those for CPA.9.086.H4(S241P). In some embodiments, the anti-TIGIT antibodies with CDRs identical to those shown in FIG. 3 (i.e., CDRs identical to those for CPA.9.086.H4(S241P) are administered at a dosage of 1 mg/kg of the anti-TIGIT antibodies with CDRs identical to those shown in FIG. 3 (i.e., CDRs identical to those for CPA.9.086.H4(S241P). In some embodiments, the anti-TIGIT antibodies with CDRs identical to those shown in FIG. 3 (i.e., CDRs identical to those for CPA.9.086.H4(S241P) are administered at a dosage of 2 mg/kg of the anti-TIGIT antibodies with CDRs identical to those shown in FIG. 3 (i.e., CDRs identical to those for CPA.9.086.H4(S241P). In some embodiments, the anti-TIGIT antibodies with CDRs identical to those shown in FIG. 3 (i.e., CDRs identical to those for CPA.9.086.H4(S241P) are administered at a dosage of 3 mg/kg of the anti-TIGIT antibodies with CDRs identical to those shown in FIG. 3 (i.e., CDRs identical to those for CPA.9.086.H4(S241P). In some embodiments, the anti-TIGIT antibodies with CDRs identical to those shown in FIG. 3 (i.e., CDRs identical to those for CPA.9.086.H4(S241P) are administered at a dosage of 4 mg/kg of the anti-TIGIT antibodies with CDRs identical to those shown in FIG. 3 (i.e., CDRs identical to those for CPA.9.086.H4(S241P). In some embodiments, the anti-TIGIT antibodies with CDRs identical to those shown in FIG. 3 (i.e., CDRs identical to those for CPA.9.086.H4(S241P) are administered at a dosage of 5 mg/kg of the anti-TIGIT antibodies with CDRs identical to those shown in FIG. 3 (i.e., CDRs identical to those for CPA.9.086.H4(S241P). In some embodiments, the anti-TIGIT antibodies with CDRs identical to those shown in FIG. 3 (i.e., CDRs identical to those for CPA.9.086.H4(S241P) are administered at a dosage of 6 mg/kg of the anti-TIGIT antibodies with CDRs identical to those shown in FIG. 3 (i.e., CDRs identical to those for CPA.9.086.H4(S241P). In some embodiments, the anti-TIGIT antibodies with CDRs identical to those shown in FIG. 3 (i.e., CDRs identical to those for CPA.9.086.H4(S241P) are administered at a dosage of 7 mg/kg of the anti-TIGIT antibodies with CDRs identical to those shown in FIG. 3 (i.e., CDRs identical to those for CPA.9.086.H4(S241P). In some embodiments, the anti-TIGIT antibodies with CDRs identical to those shown in FIG. 3 (i.e., CDRs identical to those for CPA.9.086.H4(S241P) are administered at a dosage of 8 mg/kg of the anti-TIGIT antibodies with CDRs identical to those shown in FIG. 3 (i.e., CDRs identical to those for CPA.9.086.H4(S241P). In some embodiments, the anti-TIGIT antibodies with CDRs identical to those shown in FIG. 3 (i.e., CDRs identical to those for CPA.9.086.H4(S241P) are administered at a dosage of 9 mg/kg of the anti-TIGIT antibodies with CDRs identical to those shown in FIG. 3 (i.e., CDRs identical to those for CPA.9.086.H4(S241P). In some embodiments, the anti-TIGIT antibodies with CDRs identical to those shown in FIG. 3 (i.e., CDRs identical to those for CPA.9.086.H4(S241P) are administered at a dosage of 10 mg/kg of the anti-TIGIT antibodies with CDRs identical to those shown in FIG. 3 (i.e., CDRs identical to those for CPA.9.086.H4(S241P).

In some embodiments, the anti-TIGIT antibodies with CDRs identical to those shown in FIG. 3 (i.e., CDRs identical to those for CPA.9.086.H4(S241P) is administered at a dosage of about 0.01 mg/kg to about 10 mg/kg of the anti-TIGIT antibody. In some embodiments, the anti-TIGIT antibodies with CDRs identical to those shown in FIG. 3 (i.e., CDRs identical to those for CPA.9.086.H4(S241P) is administered at a dosage of about 10 mg/kg each 3 weeks. In some embodiments, the anti-TIGIT antibodies with CDRs identical to those shown in FIG. 3 (i.e., CDRs identical to those for CPA.9.086.H4(S241P) is administered at a dosage of about 0.1 mg/kg to about 10 mg/kg of the anti-TIGIT antibody. In some embodiments, he anti-TIGIT antibodies with CDRs identical to those shown in FIG. 3 (i.e., CDRs identical to those for CPA.9.086.H4(S241P) is administered at a dosage of about 1 mg/kg to about 10 mg/kg of the anti-TIGIT antibody. In some embodiments, the anti-TIGIT antibodies with CDRs identical to those shown in FIG. 3 (i.e., CDRs identical to those for CPA.9.086.H4(S241P) is administered at a dosage of about 2 mg/kg to about 10 mg/kg of the anti-TIGIT antibody. In some embodiments, the anti-TIGIT antibodies with CDRs identical to those shown in FIG. 3 (i.e., CDRs identical to those for CPA.9.086.H4(S241P) is administered at a dosage of about 3 mg/kg to about 10 mg/kg of the anti-TIGIT antibody. In some embodiments, the anti-TIGIT antibodies with CDRs identical to those shown in FIG. 3 (i.e., CDRs identical to those for CPA.9.086.H4(S241P) is administered at a dosage of about 4 mg/kg to about 10 mg/kg of the anti-TIGIT antibody. In some embodiments, the anti-TIGIT antibodies with CDRs identical to those shown in FIG. 3 (i.e., CDRs identical to those for CPA.9.086.H4(S241P) is administered at a dosage of about 5 mg/kg to about 10 mg/kg of the anti-TIGIT antibody. In some embodiments, the anti-TIGIT antibodies with CDRs identical to those shown in FIG. 3 (i.e., CDRs identical to those for CPA.9.086.H4(S241P) is administered at a dosage of about 5 mg/kg to about 10 mg/kg of the anti-TIGIT antibody. In some embodiments, the anti-TIGIT antibodies with CDRs identical to those shown in FIG. 3 (i.e., CDRs identical to those for CPA.9.086.H4(S241P) is administered at a dosage of about 7 mg/kg to about 10 mg/kg of the anti-TIGIT antibody. In some embodiments, the anti-TIGIT antibodies with CDRs identical to those shown in FIG. 3 (i.e., CDRs identical to those for CPA.9.086.H4(S241P) is administered at a dosage of about 8 mg/kg to about 10 mg/kg of the anti-TIGIT antibody. In some embodiments, the anti-TIGIT antibodies with CDRs identical to those shown in FIG. 3 (i.e., CDRs identical to those for CPA.9.086.H4(S241P) is administered at a dosage of about 9 mg/kg to about 10 mg/kg of the anti-TIGIT antibody. In some embodiments, the anti-TIGIT antibodies with CDRs identical to those shown in FIG. 3 (i.e., CDRs identical to those for CPA.9.086.H4(S241P) is administered at a dosage of about 0.01 mg/kg, 0.03 mg/kg, 0.1 mg/kg, 0.3 mg/kg, 1 mg/kg, 3 mg/kg, or 10 mg/kg o of the anti-TIGIT antibody.

### A. Selected Dosing

In some embodiments, the present invention provides that the anti-TIGIT antibodies with CDRs identical to those shown in FIG. 3 (i.e., CDRs identical to those for CPA.9.086.H4(S241P) is administered at a dosage of about 0.01 mg/kg, 0.03 mg/kg, 0.1 mg/kg, 0.3 mg/kg, 1 mg/kg, 3 mg/kg, 10 mg/kg. In some embodiments, the anti-TIGIT antibodies with CDRs identical to those shown in FIG. 3 (i.e., CDRs identical to those for CPA.9.086.H4(S241P) is administered every 1 week, 2 weeks, 3 weeks, or 4 weeks. In some embodiments, the anti-TIGIT antibodies with CDRs identical to those shown in FIG. 3 (i.e., CDRs identical to those for CPA.9.086.H4(S241P) is administered every 3 weeks. In some embodiments, the anti-TIGIT antibodies with CDRs identical to those shown in FIG. 3 (i.e., CDRs identical to those for CPA.9.086.H4(S241P) is formulated for a unit dosage administration. In some embodiments, the anti-TIGIT antibodies with CDRs identical to those shown in FIG. 3 (i.e., CDRs identical to those for CPA.9.086.H4(S241P) is formulated for a unit dosage administration of about 0.01 mg/kg, 0.03 mg/kg, 0.1 mg/kg, 0.3 mg/kg, 1 mg/kg, 3 mg/kg, 10 mg/kg for every 1 week, 2 weeks, 3 weeks, or 4 weeks. In some embodiments, the anti-TIGIT antibodies with CDRs identical to those shown in FIG. 3 (i.e., CDRs identical to those for CPA.9.086.H4(S241P) is formulated for a unit dosage administration of about 0.01 mg/kg, 0.03 mg/kg, 0.1 mg/kg, 0.3 mg/kg, 1 mg/kg, 3 mg/kg, 10 mg/kg for every 3 weeks.

In some embodiments, the anti-TIGIT antibodies with CDRs identical to those shown in FIG. 3 (i.e., CDRs identical to those for CPA.9.086.H4(S241P) is administered at a dosage of about 0.01 mg/kg, 0.03 mg/kg, 0.1 mg/kg, 0.3 mg/kg, 1 mg/kg, 3 mg/kg, or 10 mg/kg of the anti-TIGIT antibody. In some embodiments, the CPA.9.086.H4(S241P) is administered at a dosage of about 0.01 mg/kg, 0.03 mg/kg, 0.1 mg/kg, 0.3 mg/kg, 1 mg/kg, 3 mg/kg, or 10 mg/kg.

In some embodiments, the anti-TIGIT antibodies with CDRs identical to those shown in FIG. 3 (i.e., CDRs identical to those for CPA.9.086.H4(S241P) is administered at a dosage of about 0.01 mg/kg, 0.03 mg/kg, 0.1 mg/kg, 0.3 mg/kg, 1 mg/kg, 3 mg/kg, or 10 mg/kg of the anti-TIGIT antibodies with CDRs identical to those shown in FIG. 3 (i.e., CDRs identical to those for CPA.9.086.H4(S241P) every 1 week, 2 weeks, 3 weeks, or 4 weeks. In some embodiments, the CPA.9.086.H4(S241P) is administered at a dosage of about 0.01 mg/kg, 0.03 mg/kg, 0.1 mg/kg, 0.3 mg/kg, 1 mg/kg, 3 mg/kg, or 10 mg/kg of every 1 week, 2 weeks, 3 weeks, or 4 weeks.

In some embodiments, the anti-TIGIT antibodies with CDRs identical to those shown in FIG. 3 (i.e., CDRs identical to those for CPA.9.086.H4(S241P) is administered about 10 mg/kg every 3 weeks. In some embodiments, the CPA.9.086.H4(S241P) is administered about 10 mg/kg every 3 weeks. In some embodiments, the CPA.9.086.H4(S241P) is administered about 3 mg/kg every 3 weeks.

### VII. Methods of Using the Anti-Tigit Antibodies

### A. Therapeutic Uses

The anti-TIGIT antibodies with CDRs identical to those shown in FIG. 3 (i.e., CDRs identical to those for CPA.9.086.H4(S241P) find use in treating patients, such as human subjects, generally with a condition associated with TIGIT. The term “treatment” as used herein, refers to both therapeutic treatment and prophylactic or preventative measures, which in this example relates to treatment of cancer; however, also as described below, uses of antibodies and pharmaceutical compositions are also provided for treatment of infectious disease, sepsis, and/or autoimmune conditions, and/or for inhibiting an undesirable immune activation that follows gene therapy. Those in need of treatment include those already with cancer as well as those in which the cancer is to be prevented. Hence, the mammal to be treated herein may have been diagnosed as having the cancer or may be predisposed or susceptible to the cancer. As used herein the term “treating” refers to preventing, delaying the onset of, curing, reversing, attenuating, alleviating, minimizing, suppressing, halting the deleterious effects or stabilizing of discernible symptoms of the above-described cancerous diseases, disorders or conditions. It also includes managing the cancer as described above. By “manage” it is meant reducing the severity of the disease, reducing the frequency of episodes of the disease, reducing the duration of such episodes, reducing the severity of such episodes, slowing/reducing cancer cell growth or proliferation, slowing progression of at least one symptom, amelioration of at least one measurable physical parameter and the like. For example, immunostimulatory anti-TIGIT immune molecules should promote T cell or NK or cytokine immunity against target cells, e.g., cancer, infected or pathogen cells and thereby treat cancer or infectious diseases by depleting the cells involved in the disease condition. Conversely, immunoinhibitory anti-TIGIT immune molecules should reduce T cell or NK activity and/or or the secretion of proinflammatory cytokines which are involved in the disease pathology of some immune disease such as autoimmune, inflammatory or allergic conditions and thereby treat or ameliorate the disease pathology and tissue destruction that may be associated with such conditions (e.g., joint destruction associated with rheumatoid arthritis conditions).

The anti-TIGIT antibodies with CDRs identical to those shown in FIG. 3 (i.e., CDRs identical to those for CPA.9.086.H4(S241P) are provided in therapeutically effective dosages. A “therapeutically effective dosage” of an anti-TIGIT antibodies with CDRs identical to those shown in FIG. 3 (i.e., CDRs identical to those for CPA.9.086.H4(S241P) according to at least some embodiments of the present invention preferably results in a decrease in severity of disease symptoms, an increase in frequency and duration of disease symptom-free periods, an increase in lifespan, disease remission, or a prevention or reduction of impairment or disability due to the disease affliction. For example, for the treatment of TIGIT positive tumors, a “therapeutically effective dosage” preferably inhibits cell growth or tumor growth by at least about 20%, more preferably by at least about 40%, even more preferably by at least about 60%, and still more preferably by at least about 80% relative to untreated subjects. The ability of a compound to inhibit tumor growth can be evaluated in an animal model system predictive of efficacy in human tumors. Alternatively, this property of a composition can be evaluated by examining the ability of the compound to inhibit, such inhibition in vitro by assays known to the skilled practitioner. A therapeutically effective amount of a therapeutic compound can decrease tumor size, or otherwise ameliorate symptoms in a subject.

One of ordinary skill in the art would be able to determine a therapeutically effective amount based on such factors as the subject's size, the severity of the subject's symptoms, and the particular composition or route of administration selected.

### 1. Cancer Treatment

“Cancer therapy” herein refers to any method that prevents or treats cancer or ameliorates one or more of the symptoms of cancer. Typically such therapies will comprise administration of immunostimulatory anti-TIGIT antibodies with CDRs identical to those shown in FIG. 3 (i.e., CDRs identical to those for CPA.9.086.H4(S241P) either alone or in combination with chemotherapy or radiotherapy or other biologics and for enhancing the activity thereof, i.e., in individuals wherein expression of TIGIT suppresses antitumor responses and the efficacy of chemotherapy or radiotherapy or biologic efficacy.

The anti-TIGIT antibodies with CDRs identical to those shown in FIG. 3 (i.e., CDRs identical to those for CPA.9.086.H4(S241P) of the invention find particular use in the treatment of cancer. In general, the antibodies of the invention are immunomodulatory, in that rather than directly attack cancerous cells, the a anti-TIGIT antibodies with CDRs identical to those shown in FIG. 3 (i.e., CDRs identical to those for CPA.9.086.H4(S241P) stimulate the immune system, generally by inhibiting the action of TIGIT. Thus, unlike tumor-targeted therapies, which are aimed at inhibiting molecular pathways that are crucial for tumor growth and development, and/or depleting tumor cells, cancer immunotherapy is aimed to stimulate the patient's own immune system to eliminate cancer cells, providing long-lived tumor destruction. Various approaches can be used in cancer immunotherapy, among them are therapeutic cancer vaccines to induce tumor-specific T cell responses, and immunostimulatory antibodies (i.e. antagonists of inhibitory receptors=immune checkpoints) to remove immunosuppressive pathways.

Clinical responses with targeted therapy or conventional anti-cancer therapies tend to be transient as cancer cells develop resistance, and tumor recurrence takes place. However, the clinical use of cancer immunotherapy in the past few years has shown that this type of therapy can have durable clinical responses, showing dramatic impact on long term survival. However, although responses are long term, only a small number of patients respond (as opposed to conventional or targeted therapy, where a large number of patients respond, but responses are transient).

By the time a tumor is detected clinically, it has already evaded the immune-defense system by acquiring immunoresistant and immunosuppressive properties and creating an immunosuppressive tumor microenvironment through various mechanisms and a variety of immune cells.

Accordingly, the anti-TIGIT antibodies with CDRs identical to those shown in FIG. 3 (i.e., CDRs identical to those for CPA.9.086.H4(S241P) are useful in treating cancer. Due to the nature of an immuno-oncology mechanism of action, TIGIT does not necessarily need to be overexpressed on or correlated with a particular cancer type; that is, the goal is to have the anti-TIGIT antibodies with CDRs identical to those shown in FIG. 3 (i.e., CDRs identical to those for CPA.9.086.H4(S241P) de-suppress T cell and NK cell activation, such that the immune system will go after the cancers.

“Cancer,” as used herein, refers broadly to any neoplastic disease (whether invasive or metastatic) characterized by abnormal and uncontrolled cell division causing malignant growth or tumor (e.g., unregulated cell growth). The term “cancer” or “cancerous” as used herein should be understood to encompass any neoplastic disease (whether invasive, non-invasive or metastatic) which is characterized by abnormal and uncontrolled cell division causing malignant growth or tumor, non-limiting examples of which are described herein. This includes any physiological condition in mammals that is typically characterized by unregulated cell growth.

In some embodiments, the anti-TIGIT antibodies with CDRs identical to those shown in FIG. 3 (i.e., CDRs identical to those for CPA.9.086.H4(S241P) can be used in the treatment of solid tumors (including, for example, cancers of the lung, liver, breast, brain, GI tract) and blood cancers (including for example, leukemia and preleukemic disorders, lymphoma, plasma cell disorders) carcinoma, lymphoma, blastoma, sarcoma, and leukemia or lymphoid malignancies. In some embodiments, the cancer is early. In some embodiments, the cancer is advanced (including metastatic). In some embodiments, the cancers amenable for treatment of the invention include cancers that express or do not express TIGIT and further include non-metastatic or non-invasive, as well as invasive or metastatic cancers, including cancers where TIGIT expression by immune, stromal, or diseased cells suppresses antitumor responses and anti-invasive immune responses. In some embodiments, the anti-TIGIT antibodies with CDRs identical to those shown in FIG. 3 (i.e., CDRs identical to those for CPA.9.086.H4(S241P) can be used for the treatment of vascularized tumors. In some embodiments, the cancer for treatment using the anti-TIGIT antibodies with CDRs identical to those shown in FIG. 3 (i.e., CDRs identical to those for CPA.9.086.H4(S241P) of the present invention includes carcinoma, lymphoma, sarcoma, and/or leukemia. In some embodiments, the cancer for treatment using the anti-TIGIT antibodies with CDRs identical to those shown in FIG. 3 (i.e., CDRs identical to those for CPA.9.086.H4(S241P) includes melanoma, non-melanoma skin cancer (squamous and basal cell carcinoma), mesothelioma, squamous cell cancer, lung cancer (including small-cell lung cancer, non-small cell lung cancer, soft-tissue sarcoma, Kaposi's sarcoma, adenocarcinoma of the lung, squamous carcinoma of the lung, cancer of the peritoneum, esophageal cancer, hepatocellular cancer, liver cancer (including HCC), gastric cancer, stomach cancer (including gastrointestinal cancer), pancreatic cancer, glioblastoma, cervical cancer, ovarian cancer, urothelial cancer, bladder cancer, hepatoma, glioma, brain cancer (as well as edema, such as that associated with brain tumors), breast cancer (including, for example, triple negative breast cancer), testis cancer, testicular germ cell tumors, colon cancer, colorectal cancer (CRC), colorectal cancer MSS (MSS-CRC; including refractory MSS colorectal; MSS=microsatellite stable status), primary peritoneal cancer, microsatellite stable primary peritoneal cancer, platinum resistant microsatellite stable primary peritoneal cancer, CRC (MSS unknown), rectal cancer, endometrial cancer (including endometrial carcinoma), uterine carcinoma, salivary gland carcinoma, kidney cancer, renal cell cancer (RCC), renal cell carcinoma (RCC), prostate cancer, vulval cancer, thyroid cancer, hepatic carcinoma, carcinoid carcinoma, head and neck cancer, B-cell lymphoma (including non-Hodgkin's lymphoma, as well as low grade/follicular non-Hodgkin's lymphoma (NHL), small lymphocytic (SL) NHL, intermediate grade/follicular NHL, intermediate grade diffuse NHL, Diffuse Large B cell lymphoma, high grade immunoblastic NHL, high grade lymphoblastic NHL, high grade small non-cleaved cell NHL, bulky disease NHL, mantle cell lymphoma, AIDS-related lymphoma, and Waldenström's Macroglobulinemia), Hodgkin's lymphoma (HD), chronic lymphocytic leukemia (CLL), acute lymphoblastic leukemia (ALL), T cell Acute Lymphoblastic Leukemia (T-ALL), Acute myeloid leukemia (AML), Hairy cell leukemia, chronic myeloblastic leukemia, multiple myeloma, post-transplant lymphoproliferative disorder (PTLD), abnormal vascular proliferation associated with phakomatoses, Meigs' syndrome, Merkel Cells cancer, MSI-high cancer, KRAS mutant tumors, adult T-cell leukemia/lymphoma, adenoid cystic cancer (including adenoid cystic carcinoma), malignant melanoma, pancreatic cancer, pancreatic adenocarcinoma, ovarian cancer (including ovarian carcinoma), pleural mesothelioma, neuroendocrine lung cancer (including pleural mesothelioma, neuroendocrine lung carcinoma), NSCLC large cell adenocarcinoma, non-small cell lung carcinoma (NSCLC), NSCLC squamous cell, cervical squamous cell carcinoma (cervical SCC), anal squamous cell carcinoma (anal SCC), neuroendocrine lung carcinoma, carcinoma of unknown primary, gallbladder cancer, malignant melanoma, pleural mesothelioma, multiple myeloma, gastroesophageal junction cancer, and/or Myelodysplastic syndromes (MDS).

In some embodiments, the cancer for treatment using the anti-TIGIT antibodies with CDRs identical to those shown in FIG. 3 (i.e., CDRs identical to those for CPA.9.086.H4(S241P) includes a cancer selected from the group consisting of prostate cancer, liver cancer (HCC), colorectal cancer (CRC), colorectal cancer MSS (MSS-CRC; including refractory MSS colorectal), CRC (MSS unknown), ovarian cancer (including ovarian carcinoma), endometrial cancer (including endometrial carcinoma) breast cancer, pancreatic cancer, stomach cancer, cervical cancer, head and neck cancer, thyroid cancer, testis cancer, urothelial cancer, lung cancer, melanoma, non-melanoma skin cancer (squamous and basal cell carcinoma), glioma, renal cell cancer (RCC), renal cell carcinoma (RCC), lymphoma (non-Hodgkins' lymphoma (NHL) and Hodgkin's lymphoma (HD)), Acute myeloid leukemia (AML), T cell Acute Lymphoblastic Leukemia (T-ALL), Diffuse Large B cell lymphoma, testicular germ cell tumors, mesothelioma, esophageal cancer, triple negative breast cancer, Merkel Cells cancer, MSI-high cancer, KRAS mutant tumors, adult T-cell leukemia/lymphoma, pleural mesothelioma, anal SCC, small cell lung cancer, adenocarcinoma of the lung, neuroendocrine lung cancer (including neuroendocrine lung carcinoma), HNSCC, NSCLC, NSCLC large cell, NSCLC squamous cell, cervical SCC, malignant melanoma, pancreatic cancer, pancreatic adenocarcinoma, NSCLC, adenoid cystic cancer (including adenoid cystic carcinoma), primary peritoneal cancer, microsatellite stable primary peritoneal cancer, platinum resistant microsatellite stable primary peritoneal cancer, PD1 refractory or relapsing, multiple myeloma, gastroesophageal junction cancer, and/or Myelodysplastic syndromes (MDS).

In some embodiments, the cancer for treatment using the anti-TIGIT antibodies with CDRs identical to those shown in FIG. 3 (i.e., CDRs identical to those for CPA.9.086.H4(S241P) include advanced cancer, ovarian cancer, lung cancer, non-small-cell lung carcinoma (NSCLC), PD1 refractory or relapsing NSCLC, colon cancer, colorectal cancer (CRC), MSS-CRC, plasma cell neoplasm, head and neck squamous cell carcinomas (HNSCC), PD1 refractory or relapsing HNSCC, and breast cancer.

### EXAMPLES

### Example 1: Stable Pharmaceutical Formulation CPA.9.086.H4(S241P)

This example describes the characteristics and preparation of a stable pharmaceutical formulation comprising:
(a) about 20 mg/mL of an anti-TIGIT antibody, the anti-TIGIT antibody comprising:
   i) a heavy chain from CPA.9.086.H4(S241P) (SEQ ID NO:5); and
   ii) a light chain from the light chain of CPA.9.086.H4(S241P) (SEQ ID NO: 10);
(b) about 10 mM histidine;
(c) about 30 mM L-Arginine;
(d) about 10 mM L-methionine;
(e) about 8% (w/v) sucrose; and
(f) about 75 ppm polysorbate 80,
wherein the pharmaceutical formulation has a pH of 5.4+/−0.2, was prepared.

A formulation examination of CPA.9.086.H4(S241P) in phosphate buffer at pH 7.0 proved the solubility of the antibody to be >20 mg/mL. Thus, the formulation of CPA.9.086.H4(S241P) with a target antibody concentration of 20 mg/mL was studied.

The formulation was provided with antibody concentration of 20 mg/mL and with a target pH of 5.4 and prepared for further packaging. For administration to patients, CPA.9.086.H4(S241P) would be diluted with 0.9% sodium chloride and placed into an appropriate administration container.

Stability of CPA.9.086.H4(S241P) was observed in the formulation, which contained 30 mM L-arginine, 10 mM L-histidine, 10 mM L-methionine, 8% (w/v) sucrose and 75 ppm polysorbate 80 at pH 5.4 (range: 5.4±0.2). While L-histidine was used as a buffer component, L-arginine, L-methionine, sucrose and polysorbate 80 were used to stabilize the protein and prevent self-association and aggregation. In addition, L-methionine served as an antioxidant and polysorbate 80 acts as surfactant.

Table 1 provides the formulation components. The listed amount per vial is based on an actual target fill volume of 5.3 mL, which is equivalent to a target fill weight of 5.5 g/vial. The nominal amount of CPA.9.086.H4(S241P) per vial is 100 mg.

TABLE 1
CPA.9.086.H4(S241P) Formulation Components

| Amount | |
|---|---|
| Nominal* | Actual** |
| [mg/vial] | [mg/vial] |

Active Ingredient

| | | | | | |
|---|---|---|---|---|---|
| CPA.9.086 | active | 20 | mg/mL | 100.0 | 106.0 |
| (S241P)) mAb | ingredient | | | | |

Excipients

| | | | | | |
|---|---|---|---|---|---|
| Sucrose | stabilizer | 8% | w/v | 400.0 | 424.0 |
| L-Arginine HCl | stabilizer | 30 | mM | 31.6 | 33.5 |
| L-Histidine | buffer agent | 10 | mM | 1.15 | 1.22 |
| L-Histidine HCl | buffer agent | | | 8.90 | 9.43 |
| monohydrate | | | | | |
| L-Methionine | stabilizer | 10 | mM | 7.45 | 7.90 |
| | (antioxidant) | | | | |
| Polysorbate 80 | stabilizer | 75 | ppm | 0.38 | 0.40 |
| | (surfactant) | | | | |

*amount based on a nominal fill volume of 5.0 mL
**amount based on an actual target fill volume of 5.3 mL/vial (=a target fill weight of 5.5 g/vial)

The temperature stability was determined based on the following:
Liquid stability samples 5° C. and 25° C. for 0.5, 1, 2 and 6 months
Lyophilized stability samples 5° C. and 25° C. for 1, 3, and 6 months

Data obtained indicated that CPA.9.086.H4(S241P) was stable at 5° C. both in liquid and lyophilized formulations during storage at least for 6 months. At 25° C. CPA.9.086.H4(S241P) exhibited more stability in the lyophilized formulation.

Stability was also determined for samples based on freeze/thaw, shaking, aggregation, fragmentation, and other standard analyses and was determined to be stable. The stable formulation also maintained biological function for CPA.9.086.H4(S241P).

### CONCLUSION

CPA.9.086.H4(S241P) was stable in both liquid and lyophilized formulations in terms of physical appearance, formation of aggregate, and other properties for at least for 6 months at 5° C. Additional results further indicated that CPA.9.086.H4(S241P) was stable in terms of physical appearance, formation of aggregates, fragments, and basic/acidic species under repeated freeze/thaw, shaking, mixing, and filtration processes.

The examples set forth above are provided to give those of ordinary skill in the art a complete disclosure and description of how to make and use the embodiments of the compositions, systems and methods of the invention, and are not intended to limit the scope of what the inventors regard as their invention. Modifications of the above-described modes for carrying out the invention that are obvious to persons of skill in the art are intended to be within the scope of the following claims. All patents and publications mentioned in the specification are indicative of the levels of skill of those skilled in the art to which the invention pertains.

All headings and section designations are used for clarity and reference purposes only and are not to be considered limiting in any way. For example, those of skill in the art will appreciate the usefulness of combining various aspects from different headings and sections as appropriate according to the spirit and scope of the invention described herein.

All references cited herein are hereby incorporated by reference herein in their entireties and for all purposes to the same extent as if each individual publication or patent or patent application was specifically and individually indicated to be incorporated by reference in its entirety for all purposes.

Many modifications and variations of this application can be made without departing from its spirit and scope, as will be apparent to those skilled in the art. The specific embodiments and examples described herein are offered by way of example only, and the application is to be limited only by the terms of the appended claims, along with the full scope of equivalents to which the claims are entitled.

1. A stable liquid pharmaceutical formulation of an anti-TIGIT antibody comprising:
(a) an anti-TIGIT antibody, the anti-TIGIT antibody comprising:
i) a heavy chain variable domain comprising the vhCDR1, vhCDR2, and vhCDR3 from the heavy chain of CPA.9.086.H4(S241P) (SEQ ID NO:5); and
ii) a light chain variable domain comprising the vlCDR1, vlCDR2, and vlCDR3 from the light chain of CPA.9.086.H4(S241P) (SEQ ID NO:10);
(b) from 8 mM to 12 mM histidine;
(c) from 25 mM to 35 mM L-Arginine;
(d) from 8 mM to 12 mM L-methionine;
(e) from 7% (w/v) to 9% (w/v) sucrose; and
(f) from 70 ppm to 80 ppm polysorbate 80,
wherein the pharmaceutical formulation has a pH of 5.4+/−0.2.

2. The stable liquid pharmaceutical formulation according to claim 1, wherein said anti-TIGIT antibody comprises a CH1-hinge-CH2-CH3 sequence of IgG4, wherein said hinge region optionally comprises mutations.

3. (canceled)

4. The stable liquid pharmaceutical according to claim 1, wherein the heavy chain variable domain is from the heavy chain of CPA.9.086.H4(S241P) (SEQ ID NO: 5) and the light chain variable domain is from the light chain of CPA.9.086.H4(S241P) (SEQ ID NO: 10).

5. The stable liquid pharmaceutical formulation according to claim 1, wherein said anti-TIGIT antibody comprises a CL region of human kappa 2 light chain.

6. The stable liquid pharmaceutical formulation according to claim 1, wherein said pharmaceutical formulation comprises from about 15 mg/mL to about 25 mg/mL anti-TIGIT antibody.

7. The stable liquid pharmaceutical formulation according to claim 1, wherein said pharmaceutical formulation comprises about 20 mg/mL anti-TIGIT antibody.

8. The stable liquid pharmaceutical formulation according to claim 1, wherein said pharmaceutical formulation comprises about 10 mM histidine.

9. The stable liquid pharmaceutical formulation according to claim 1, wherein said pharmaceutical formulation comprises about 30 mM L-arginine.

10. The stable liquid pharmaceutical formulation according to claim 1, wherein said pharmaceutical formulation comprises about 10 mM L-methionine.

11. The stable liquid pharmaceutical formulation according to claim 1, wherein said pharmaceutical formulation comprises about 8% sucrose.

12. The stable liquid pharmaceutical formulation according to claim 1, wherein said pharmaceutical formulation comprises about 75 ppm polysorbate 80.

13. A stable liquid pharmaceutical formulation of an anti-TIGIT antibody comprising:
(a) about 20 mg/mL of an anti-TIGIT antibody, the anti-TIGIT antibody comprising:
i) a heavy chain variable domain comprising the vhCDR1, vhCDR2, and vhCDR3 from the heavy chain of CPA.9.086.H4(S241P) (SEQ ID NO:5); and
ii) a light chain variable domain comprising the vlCDR1, vlCDR2, and vlCDR3 from the light chain of CPA.9.086.H4(S241P) (SEQ ID NO:10);
(b) about 10 mM histidine;
(c) about 30 mM L-Arginine;
(d) about 10 mM L-methionine;
(e) about 8% (w/v) sucrose; and
(f) about 75 ppm polysorbate 80,
wherein the pharmaceutical formulation has a pH of 5.4+/−0.2.

14. (canceled)

15. (canceled)

16. The stable liquid pharmaceutical formulation according to claim 13, wherein said pharmaceutical formulation is stable at 5° C. to 25° C. for at least 1 week, 2 weeks, 3 weeks, 4 weeks, 2 months, 3 months, 4 months, 5 months, 6 months, 7 months, or 8 months.

17. The stable liquid pharmaceutical formulation according to claim 13, wherein said pharmaceutical formulation is stable at 5° C. to 25° C. for at least 6 months, at least 12 months, or at least 18 months.

18. The stable liquid pharmaceutical formulation according to claim 13, wherein said pharmaceutical formulation is administered at a dosage of from about 0.01 mg/kg to about 100 mg/kg, from about 0.05 mg/kg to about 50 mg/kg, from about 0.1 mg/kg to about 20 mg/kg, or from about 1 mg/kg to about 10 mg/kg.

19. The stable liquid pharmaceutical formulation according to claim 13, wherein said pharmaceutical formulation is administered at a dosage of about 0.01 mg/kg, about 0.01 mg/kg, about 0.03 mg/kg, about 0.1 mg/kg, about 0.3 mg/kg, about 1 mg/kg, about 3 mg/kg, about 10 mg/kg, about 15 mg/kg, or about 20 mg/kg, or about 30 mg/kg.

20. The stable liquid pharmaceutical formulation according to claim 13, wherein said anti-TIGIT antibody is administered once every week, once every two weeks, once every three weeks, or once every four weeks, once every five weeks, or once every six weeks.

21. The stable liquid pharmaceutical formulation according to claim 13, wherein said anti-TIGIT antibody is administered as a single dose.

22. The stable liquid pharmaceutical formulation according to claim 13, wherein said pharmaceutical formulation is formulated for intravenous (IV) infusion.

23. A method of treatment of cancer in a patient in need thereof, comprising administering said stable liquid pharmaceutical formulation according to claim 1.

24. (canceled)

25. The method according to claim 23, wherein said cancer is selected from the group consisting of prostate cancer, liver cancer (HCC), colorectal cancer (CRC), colorectal cancer MSS (MSS-CRC; including refractory MSS colorectal), CRC (MSS unknown), ovarian cancer (including ovarian carcinoma), endometrial cancer (including endometrial carcinoma), breast cancer, pancreatic cancer, stomach cancer, cervical cancer, head and neck cancer, thyroid cancer, testis cancer, urothelial cancer, lung cancer, melanoma, non-melanoma skin cancer (squamous and basal cell carcinoma), glioma, renal cell cancer (RCC), renal cell carcinoma (RCC), lymphoma (non-Hodgkins' lymphoma (NHL) and Hodgkin's lymphoma (HD)), Acute myeloid leukemia (AML), T cell Acute Lymphoblastic Leukemia (T-ALL), Diffuse Large B cell lymphoma, testicular germ cell tumors, mesothelioma, esophageal cancer, triple negative breast cancer, Merkel Cells cancer, MSI-high cancer, KRAS mutant tumors, adult T-cell leukemia/lymphoma, pleural mesothelioma, anal SCC, small cell lung cancer, adenocarcinoma of the lung, neuroendocrine lung cancer (including neuroendocrine lung carcinoma), HNSCC, PD1 refractory or relapsing HNSCC, NSCLC, PD1 refractory or relapsing NSCLC, NSCLC large cell, NSCLC squamous cell, cervical SCC, malignant melanoma, pancreatic cancer, pancreatic adenocarcinoma, NSCLC, adenoid cystic cancer (including adenoid cystic carcinoma), primary peritoneal cancer, microsatellite stable primary peritoneal cancer, platinum resistant microsatellite stable primary peritoneal cancer, multiple myeloma, gastroesophageal junction cancer, and Myelodysplastic syndromes (MDS).

26. A stable lyophilized pharmaceutical formulation, wherein said formulation is obtained by lyophilizing the stable liquid pharmaceutical formulation according to claim 1.

27. (canceled)

28. (canceled)


SEQUENCE LISTING (14 sequences)

SEQ ID NO: 1 - protein, 121 aa, synthetic construct
EVQLVETGGGLIQPGRSLRLSCAASGFTFSSYAMHWVRQAPGKGLEWVAVISYAGEVKYYADSVKGRFTISRDNSKNTLYLQMNSLRAEDTAVYYCARDPLPLHYYGMDVWGQGTTVTVSS

SEQ ID NO: 2 - protein, 8 aa, synthetic construct
GFTFSSYA

SEQ ID NO: 3 - protein, 8 aa, synthetic construct
ISYAGEVK

SEQ ID NO: 4 - protein, 14 aa, synthetic construct
ARDPLPLHYYGMDV

SEQ ID NO: 5 - protein, 448 aa, synthetic construct
EVQLVETGGGLIQPGRSLRLSCAASGFTFSSYAMHWVRQAPGKGLEWVAVISYAGEVKYYADSVKGRFTISRDNSKNTLYLQMNSLRAEDTAVYYCARDPLPLHYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 6 - protein, 111 aa, synthetic construct
QSALTQPRSASGNPGQRVTISCSGSSSNMGRRPVNWYQQIPGTAPKLLIYSQNQRPSGVPDRFSGSQSGTSASLTISGLQSEDEAEYFCAVWDDIGRVLQLGGGTQLAVLG

SEQ ID NO: 7 - protein, 8 aa, synthetic construct
SSNMGRRP

SEQ ID NO: 8 - skipped

SEQ ID NO: 9 - protein, 11 aa, synthetic construct
AVWDDIGRVLQ

SEQ ID NO: 10 - protein, 216 aa, synthetic construct
QSALTQPRSASGNPGQRVTISCSGSSSNMGRRPVNWYQQIPGTAPKLLIYSQNQRPSGVPDRFSGSQSGTSASLTISGLQSEDEAEYFCAVWDDIGRVLQLGGGTQLAVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 11 - protein, 120 aa, synthetic construct
MMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIP

SEQ ID NO: 12 - protein, 121 aa, synthetic construct
MMTGTIETTGNISAKKGGSVILQCHLSSTMAQVTQVNWEQHDHSLLAIRNAELGWHIYPAFKDRVAPGPGLGLTLQSLTMNDTGEYFCTYHTYPDGTYRGRIFLEVLESSVAEHSARFQIP

SEQ ID NO: 13 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK